(19) United States
(12) Patent Application Publication
(10) Pub. No.: US 2005/0090006 A1
(43) Pub. Date: Apr. 28, 2005

(54) Schwann cell specific enhancers and methods of use thereof

(76) Inventors: Alan C. Peterson, Westmount Quebec (CA); David Foran, Mason, MI (US); Lorella Garofalo, Lasalle Quebec (CA); Reza Forghani, Montreal Quebec (CA); Hooman Farhadi, Montreal (CA); Pierre Lepage, Montreal (CA); Thomas J. Hudson, Montreal (CA)

Correspondence Address: Dowell & Dowell, Suite 309, 1215 Jefferson Davis Highway, Arlington, VA, 22202-3124, US

(21) Appl. No.: 10/476,389

(22) PCT Filed: May 2, 2002
(86) PCT No.: PCT/CA02/00657

Related U.S. Application Data
(60) Provisional application No. 60/290,501, filed on May 11, 2001.

(30) Foreign Application Priority Data
May 2, 2001 (CA) 2,347,583
May 3, 2001 (CA) 2,343,953

(51) Int. Cl. C12Q 1/68; C07H 21/04; C12N 15/85

(52) U.S. Cl. 435/455; 435/320.1; 435/325; 435/6; 536/23.2

(57) ABSTRACT

The present invention provides isolated nucleic acid molecules which exhibit transcriptional enhancement in Schwann cells. The present invention also provides vectors comprising the subject isolated nucleic acid molecules. Methods of regulating gene expression in Schwann cells and therapeutic uses involving the isolated nucleic acids are also provided.

### FIELD OF THE INVENTION

The invention is in the field of nucleic acid sequences capable of regulating transcription, particularly sequences that may enhance expression in Schwann cells.

### BACKGROUND OF THE INVENTION

Schwann cells arise from a common pool of neural crest cells and differentiate into myelinating or non-myelinating glia (Mirsky and Jessen, 1996). Axonal signals control the decision to myelinate quantitative features of the sheath and are required for its subsequent maintenance (Aguayo et al., 1976a; Aguayo et al., 1976b; Berthold, 1978). These axonal instructions operate, in part, by controlling the expression of the genes encoding myelin structural proteins. In PNS myelin these include protein zero (P0), peripheral myelin protein 22 (PMP22), and myelin basic protein (MBP). High-level expression of these genes coincides with myelin elaboration, continues throughout myelinogenesis and ceases should the axon be disrupted (Gupta et al., 1988; Trapp et al., 1988; Lamperth et al., 1990; LeBlanc and Poduslo, 1990; Stahl et al., 1990; Snipes et al., 1992). Molecules implicated in the control of Schwann cell maturation and myelin formation include hormones such as progesterone, the second messenger cAMP, the zinc finger transcription factor Krox-20 and the POU domain transcription factor Tst-1/SCIP/Oct-6 (SCIP) (Lemke and Chao, 1988; Monuki et al., 1989; Monuki et al., 1990; He et al., 1991; Morgan et al., 1991; Topilko et al., 1994; Koenig et al., 1995; Weinstein et al., 1995; Bermingham et al., 1996; Jaegle et al., 1996; Zorick and Lemke, 1996). However, neither the molecules that transduce axonal instructions nor the Schwann cell signaling pathways that they control are known. Similarly, the most downstream components of such pathways, the transcription factors and DNA regulatory elements that confer expression to myelin genes, remain to be elucidated.

The presence of positive Schwann cell targeting elements has been detected from P0 or CNPase 5′ flanking sequences (Messing et al., 1992; Gravel et al. 1998). However, 5′ flanking sequences of MBP have only been shown to result in expression in oligodendrocytes, the myelin forming cell type in the central nervous system (Foran and Peterson, 1992; Gow et al., 1992; Miskimins et al., 1992; Goujet-Zalc et al., 1993; Stankoff et al., 1996). There is, therefore, a need to identify the location of other Schwann cell targeting elements.

### SUMMARY OF THE INVENTION

In various aspects, the present invention provides nucleic acid molecules which modulate transcription of operably-linked sequences in Schwann cells. In some embodiments, such sequences are designated herein as SCE1 and SCE2. In some embodiments, the nucleic acid molecules enhance transcription of operably-linked sequences in Schwann cells. In other embodiments, the nucleic acid molecules repress transcription of operably-linked sequences in Schwann cells.

The invention includes recombinant nucleic acid molecules comprising at least one isolated nucleic acid molecule of the present invention that enhances transcription of operably-linked sequences in Schwann cells. Preferably, the recombinant nucleic acid molecules further comprise a heterologous promoter sequence, and a heterologous coding sequence, wherein said sequences are operably-linked.

The invention further provides a method for regulating gene expression in Schwann cells, comprising transforming a host cell with a vector comprising at least one isolated nucleic acid molecule, of the present invention, that enhances transcription of operably-linked sequences. The host cell is maintained under conditions such that an operably-linked coding sequence is expressed. The host cell may be a cell in vivo or a cell in culture. For example, the enhancer nucleic acid molecule may be used to mediate an in vitro protein production process using Schwann cells.

The invention also provides methods for gene therapy for Schwann cell-associated disease. The methods include the expression of transgenes in myelin-forming Schwann cells for the treatment of diseases of the peripheral nervous system.

In another aspect, the invention provides cells and non-human animals harbouring the recombinant nucleic acid molecules of the invention. Such animals may have a modified phenotype compared to a non-transgenic animal of the same species, such as enhanced expression of a coding sequence in Schwann cells. Methods of producing such transgenic animals are provided, for example by introducing into an animal the isolated nucleic acids of the invention.

The invention also provides a method for purifying Schwann cell-specific transcription factors, the method comprises contacting cell extracts from Schwann cells with DNA comprising the isolated nucleic acids of the invention under conditions suitable for specific binding of the transcription factor(s) to the recognition site(s) on the DNA, whereupon unbound material may be removed by washing and the retained material containing the transcription factor(s) recovered.

### BRIEF DESCRIPTION OF THE DRAWINGS

FIG. 1 shows the 1022 bp (Mod4) sequence comprising the identified enhancer sequence, SCE1, derived from human genomic DNA. The SCE1 sequence is underlined.

FIG. 2 shows the human sequence comprising Mod4 and the SCE1 enhancer sequence. The SCE1 sequence is underlined.

FIG. 3 shows the Mod4 sequence comprising the SCE1 enhancer sequence derived from mouse genomic DNA. The SCE1 sequence is underlined

FIG. 4 shows an alignment of human and mouse Mod4 sequences, wherein the upper sequence is human mbp module 4 (1022 bp) and the lower sequence is Mod4=/−1500 bp SacII.

FIG. 5 shows the alternative consensus sequences from human/mouse Mod4, wherein in one consensus sequence gaps are filled in with the human sequence and in the other gaps are filled with the mouse, showing where the consensus SCE1 sequence begins.

FIG. 6 shows the sequence comprising contiguous SCE1 and SCE2 sequences derived from mouse genomic DNA. The SCE1 sequence is underlined and the remaining 5′ sequence comprises SCE2.

FIG. 7 shows the MBP-promoted lacZ reporter constructs used to map Schwann cell enhancers in transgenic mice.

FIG. 8 shows lacZ reporter constructs comprising the SCE1 enhancer sequence operably-linked to a heterologous promoter (hsp68).

FIG. 9 shows a 486 bp mouse SCE1 sub-sequence defined by 5′ SacII and 3′ SbfI restriction sites.

FIG. 10 shows a 213 bp mouse SCE1 sub-sequence defined by SacII and AvrII restriction sites.

FIG. 11 shows a construct containing the 213 bp sequence shown in FIG. 10, plus 203 bp of contiguous 5′ mouse sequence from SCE2 (a total of 416 bps).

FIG. 12 shows the alternative consensus sub-sequences from human/mouse SCE1 derived from the mouse SCE1 sub-sequence of FIG. 9, wherein in one consensus sequence gaps are filled in with the human sequence and in the other gaps are filled with the mouse sequence.

FIG. 13 shows the alternative consensus sub-sequences from human/mouse SCE1 derived from the mouse SCE1 sub-sequence of FIG. 10, wherein in one consensus sequence gaps are filled in with the human sequence and in the other gaps are filled with the mouse sequence.

FIG. 14 shows a 510 bp SCE1 sub-sequence defined by 5′ NaeI and 3′ NaeI restriction sites.

### DETAILED DESCRIPTION OF THE INVENTION

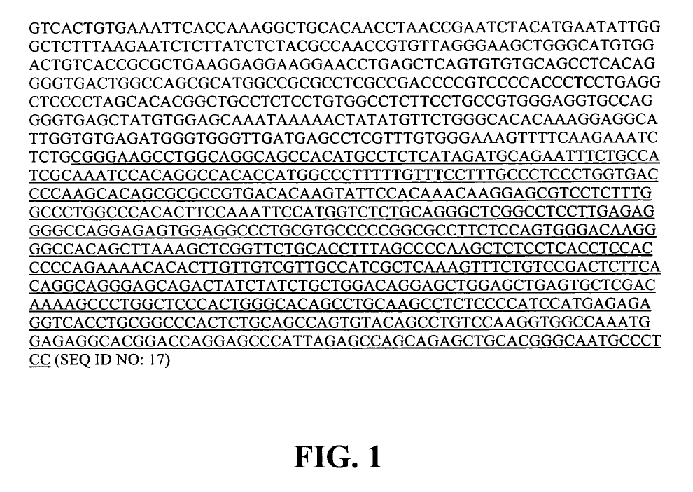

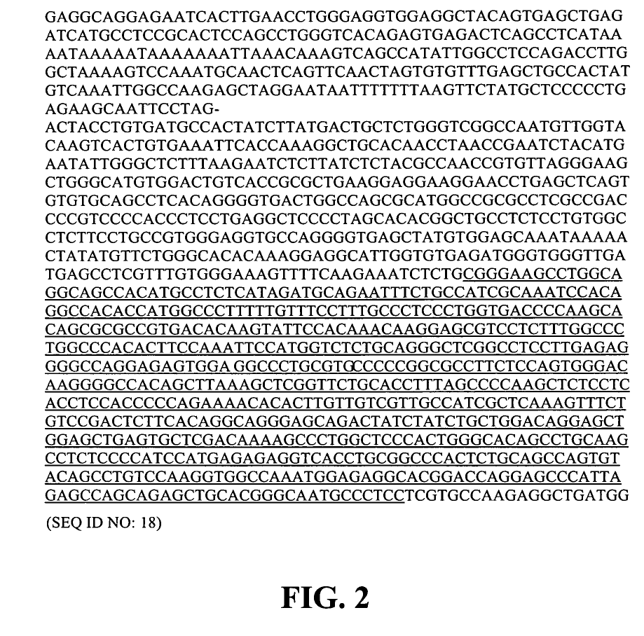

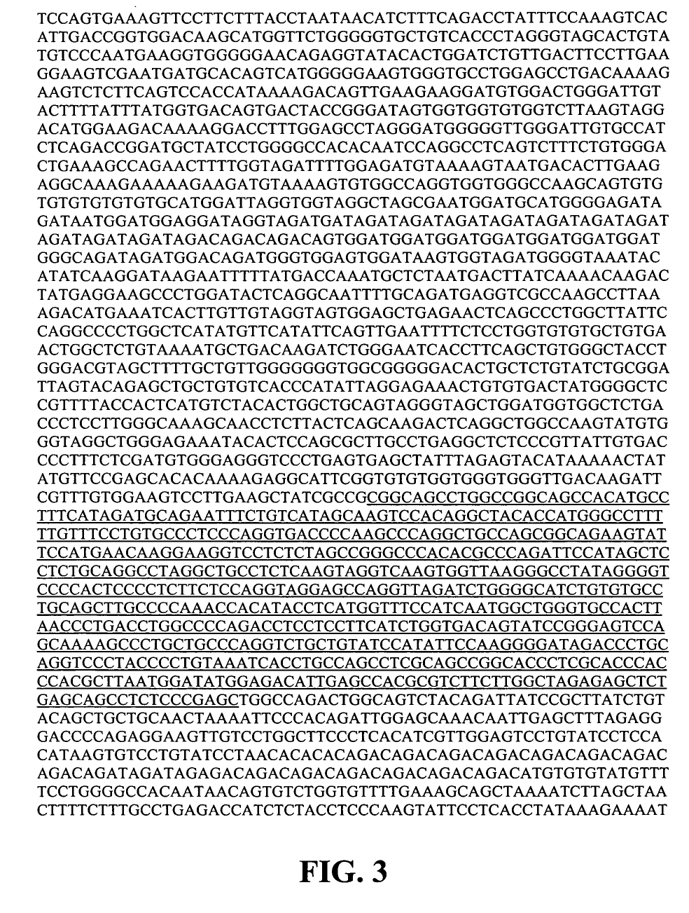

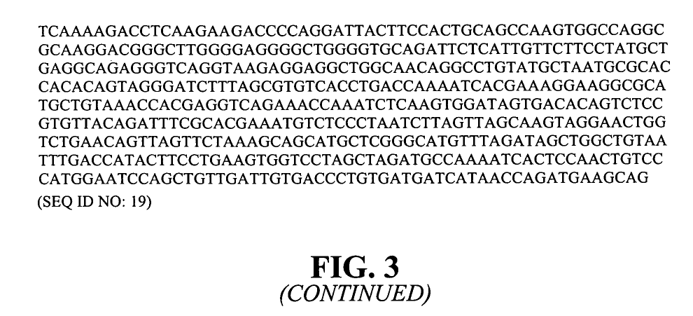

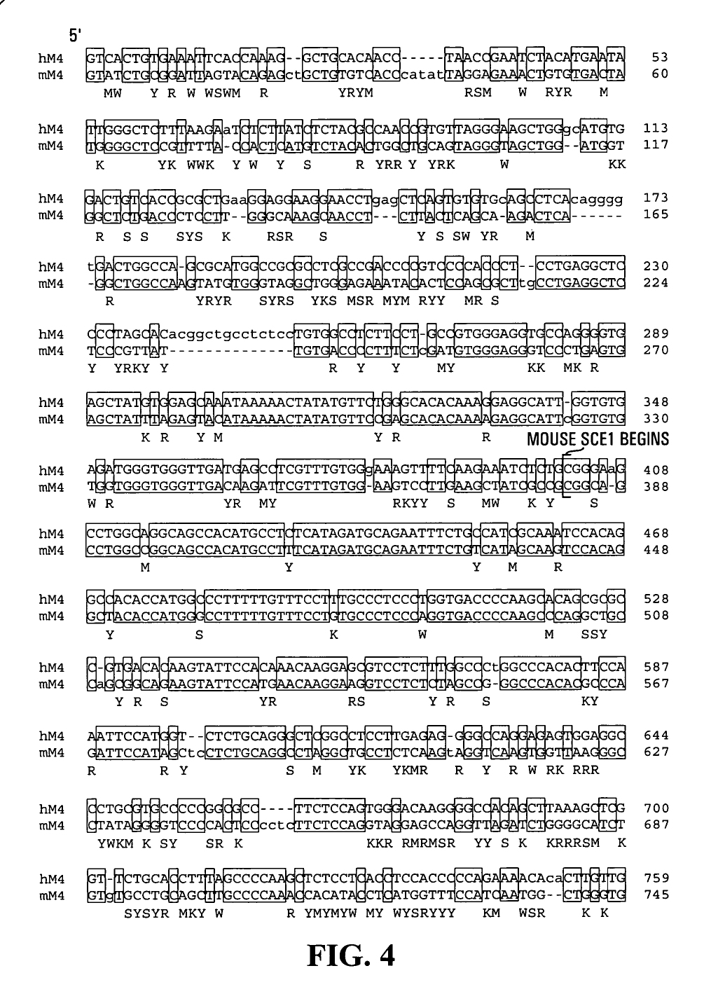

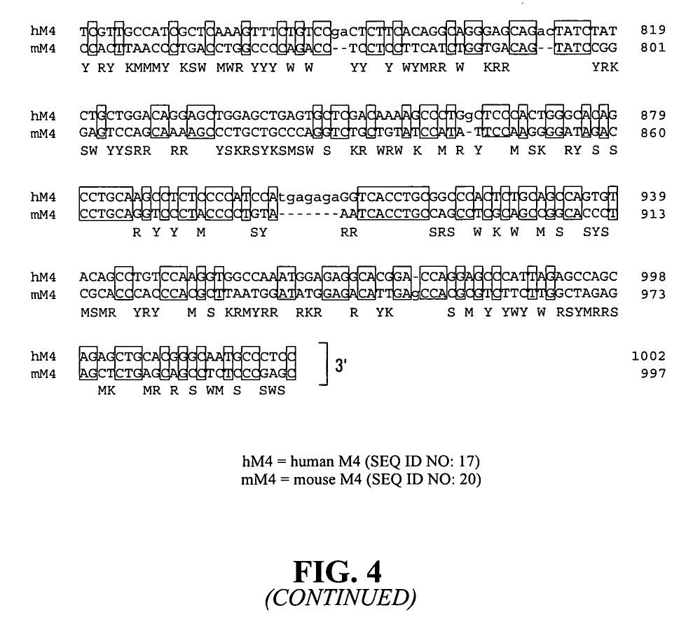

The present invention provides isolated nucleic acid molecules which regulate transcription in Schwann cells by modulating transcriptional activity. In embodiments, such modulation comprises an enhancement of transcription or an increase in transcriptional activity. In other embodiments, such modulation comprises repression of transcription or a decrease in transcriptional activity. In a preferred embodiment, the nucleic acid is the enhancer elements SCE1 and/or SCE2, as shown in FIGS. 1-6. The sequences were identified as described in the examples set forth below by their high-level reporter gene expression in myelin forming Schwann cells.

By isolated, it is meant that the isolated substance has been substantially separated or purified away from other biological components with which it would otherwise be associated, for example in vivo. The term ‘isolated’ therefore includes substances purified by standard purification methods, as well as substances prepared by recombinant expression in a host, as well as chemically synthesized substances.

The language “regulates transcription” is intended to include a mechanism which increases or decreases the production of mRNA from a particular gene. Regulation of transcription can be measured using a promoter and regulatory region fused to a gene, preferably a reporter gene whose activity is easily measured. For example, the reporter gene may include β-galactosidase. By “transciptional enhancement”, it is meant to refer to a functional property of producing an increase in the rate of transcription of linked sequences that contain a functional promoter.

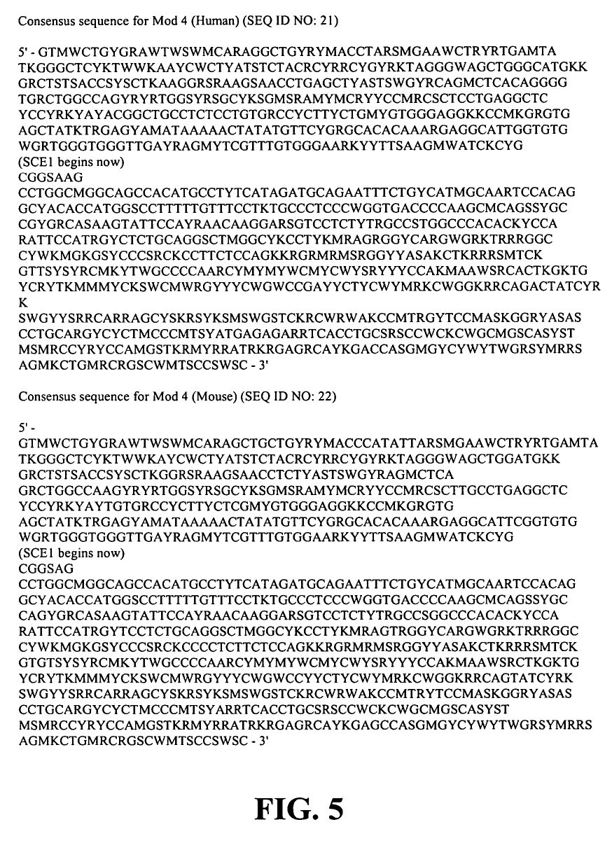

Sequences may be derived or obtainable from genomic libraries or directly from isolated DNA by deduction and synthesis based upon sequences of the present invention, such as SCE1. Derived sequences may be identified in different organisms, for example by isolation using as probes the nucleic acid sequences of the invention. Derived nucleic acids of the invention may be obtained by chemical synthesis, isolation, or cloning from genomic DNAs using techniques known in the art, such as the Polymerase Chain Reaction (PCR). Consensus sequences, such as illustrated in FIG. 5 are alternative embodiments of the nucleic acids of the invention, derived from the disclosed SCE1 sequences. Nucleic acids of the present invention may be used to design alternative primers (probes) suitable for use as PCR primers to amplify particular regions of the SCE1 sequence. Such PCR primers may for example comprise a sequence of 15-20 consecutive nucleotides of the sequences of the invention. To enhance amplification specificity, primers of 20-30 nucleotides in length may also be used. Methods and conditions for PCR amplification are described in Innis et al. (1990); Sambrook et al. (1989); and Ausubel et al. (1995). As used herein, the term “probe” when made in reference to an olignucleotide refers to an oligonucleotide which is capable of hybridizing to another oligonucleotide of interest. A probe may be single-stranded or double-stranded. Probes are, for example, useful in the detection, identification, amplification and isolation of particular gene sequences. Oligonucleotide probes may be labelled with a “reporter molecule,” so that the probe is detectable using a detection system, such as enzymatic, fluorescent, radioactive or luminescent detection systems.

Derived nucleic acids of the invention may also be identified by hybridization, such as Southern analysis. Southern analysis is a method by which the presence of DNA sequences in a target nucleic acid mixture are identified by hybridization to a labeled probe, comprising an oligonucleotide or DNA fragment of a nucleic acid of the invention. Probes for Southern analysis may for example be at least 15 nucleotides in length Southern analysis typically involves electrophoretic separation of DNA digests on agarose gels, denaturation of the DNA after electrophoretic separation, and transfer of the DNA to nitrocellulose, nylon, or another suitable membrane support for analysis with a radiolabeled, biotinylated, or enzyme-labeled probe as described in Sambrook et al. (1989).

In alternative embodiments, a nucleic acid of the invention may be at least 70% identical when optimally aligned to the SCE1 sequence. In alternative embodiments, the degree of identity may be any integer value between 50% and 100%, such as 60%, 80%, 90%, 95% or 99%. When a position in the compared sequence is occupied by the same nucleotide, following optimal alignment of the sequences, the molecules are considered to have identity at that position. The degree of identity between sequences is a function of the number of matching positions shared by the sequences. In terms of percentage, identity is the sum of identical positions, divided by the total length over which the sequences are aligned, multiplied by 100.

Various aspects of the present invention encompass nucleic acid sequences that are homologous to other sequences. As the term is used herein, a nucleic acid sequence is “homologous” to another sequence if the two sequences are substantially identical and the functional activity of the sequences is conserved (for example, both sequences have the ability to regulate transcription in Schwann cells; as used herein, the term ‘homologous’ does not infer evolutionary relatedness).

Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 80%, 90% or 95%. As used herein, a given percentage of homology between sequences denotes the degree of sequence identity in optimally aligned sequences.

Optimal alignment of sequences for comparisons of similarity may be automated using a variety of algorithms, such as the local homology algorithm of Smith and Waterman (1981) Adv. Appl. Math 2: 482, the homology alignment algorithm of Needleman and Wunsch (1970) J. Mol. Biol. 48:443, the search for similarity method of Pearson and Lipman (1988) Proc. Natl. Acad. Sci. USA 85: 2444, and the computerized implementations of these algorithms (such as GAP, BESTFIT, FASTA and TFASTA in the Wisconsin Genetics Software Package, Genetics Computer Group, Madison, Wis., U.S.A.). Sequence similarity may also be determined using the BLAST algorithm, described in Altschul et al. (1990), J. Mol. Biol. 215:403-10 (using the published default settings). Software and instructions for performing BLAST analysis may be available through the National Center for Biotechnology Information in the United States (including the programs BLASTP, BLASTN, BLASTX, TBLASTN and TBLASTX that may be available through the internet at http.//www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database (reference) sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff (1992) Proc. Natl. Acad. Sci. USA 89: 10915-10919), a gap existence cost of 11, a per residue gap cost of 1, a lambda ratio of 0.85, alignments ( ) of 50, expectation (E) of 10, M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide sequences are considered substantially identical if the smallest sum probability in a comparison of the test sequences is less than about 1, preferably less than about 0.1, more preferably less than about 0.01, and most preferably less than about 0.001. In the PSI-BLAST implementation of the BLAST algorithm, an expect value for inclusion in PSI-BLAST iteration may be 0.001 (Altschul et al. (1997), Nucleic Acids Res. 25:3389-3402). Searching parameters may be varied to obtain potentially homologous sequences from database searches.

An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in O₂×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology-Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In another aspect of the invention, the isolated nucleic acid may further be incorporated into a recombinant expression vector. Preferably, the vector will comprise an enhancer sequence of the present invention operably-linked to a promoter and a protein coding region such as MBP, a reporter gene or some other gene of interest. A first nucleic acid sequence is operably-linked with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since enhancers generally function when separated from the promoters by several kilobases and intronic sequences may be of variable lengths, some polynucleotide elements may be operably-linked but not contiguous.

The recombinant expression vector of the present invention can be constructed by standard techniques known to one of ordinary skill in the art and found, for example, in Sambrook et al. (1989) in Molecular Cloning: A Laboratory Manual. A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation and selection in bacteria and host cells. In addition, the vectors of the present invention may comprise a sequence of nucleotides for one or more restriction endonuclease sites. Coding sequences for heterologous genes, sequences for selectable markers, and reporter genes are well known to persons skilled in the art. By “heterologous genes” is meant coding sequences or parts thereof which are not naturally connected to the sequences of the invention in the same manner as in the recombinant constructs of the invention.

A recombinant expression vector comprising an enhancer sequence of the present invention may be introduced into a host cell, which may include a living cell capable of expressing the protein coding region from the defined recombinant expression vector. The living cell may include both a cultured cell and a cell within a living organism. Accordingly, the invention also provides host cells containing the recombinant expression vectors of the invention. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (Molecular Cloning: A Laboratory Manual, 2nd Edition, Cold Spring Harbor Laboratory press (1989)), and other laboratory manuals. Methods for introducing DNA into mammalian cells in vivo are also known, and may be used to deliver the vector DNA of the invention to a subject for gene therapy for diseases of the peripheral nervous system (Charcot-Marie Tooth disease) and possibly for central neurons that project axons to the periphery (ALS), for example.

A cell, tissue, organ, or organism into which has been introduced a foreign nucleic acid, is considered “transformed”, “transfected”, or “transgenic”. A transgenic or transformed cell or organism also includes progeny of the cell or organism and progeny produced from a breeding program employing a transgenic organism as a parent and exhibiting an altered phenotype resulting from the presence of a recombinant nucleic acid construct A transgenic organism is therefore an organism that has been transformed with a heterologous nucleic acid, or the progeny of such an organism that includes the transgene.

For stable transfection of mammalian cells, it is known that, depending upon the expression vector and transfection technique used, only a small fraction of cells may integrate the foreign DNA into their genome. In order to identify and select these integrants, a gene that encodes a selectable marker (such as resistance to antibiotics) may be introduced into the host cells along with the gene of interest Preferred selectable markers include those that-confer resistance to drugs, such as G418, hygromycin and methotrexate. Nucleic acids encoding a selectable marker may be introduced into a host cell on the same vector as that encoding the peptide compound or may be introduced on a separate vector. Cells stably transfected with the introduced nucleic acid may be identified by drug selection (cells that have incorporated the selectable marker gene will survive, while the other cells die).

A nucleic acid of the invention may be delivered to cells in vivo using methods such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

For use as a gene therapy vector, the genome of an adenovirus may be manipulated so that it encodes and expresses a peptide compound of the invention, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al. Curr. Topics in Micro. and Immunol. (1992) 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). An AAV vector such as that described in Tratschin et al. (1985) Mol. Cell. Biol. 5:3251-3260 may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in the invention.

General methods for gene therapy are known in the art See for example, U.S. Pat. No. 5,399,346 by Anderson et al. A biocompatible capsule for delivering genetic material is described in PCT Publication WO 95/05452 by Baetge et al. Methods of gene transfer into hematopoietic cells have also previously been reported (see Clapp, D. W., et al., Blood 78: 1132-1139 (1991); Anderson, Science 288:627-9 (2000); and, Cavazzana-Calvo et al., Science 288:669-72 (2000)).

Transcription factor(s) bind to polynucleotide sequence(s) or sequence motif(s) which are identified as being sites for the sequence-specific interaction of one or more transcription factors, frequently taking the form of direct protein-DNA binding In one embodiment of the invention, the nucleic acid may be used for purifying MBP-specific transcription factors. For example, cell extracts from Schwann cells may be contacted with DNA comprising an enhancer sequence of the present invention (SCE1), under conditions suitable for specific binding of the transcription factor(s) to the recognition site(s) on the DNA, whereupon unbound material may be removed by washing and the retained material containing the transcription factor(s) recovered. Eukaryotic transcription factors known in the art include, but are not limited to: NFAT, AP1, AP-2, Spl, OCT-I OCT-2, OAP, MFKB, CREB, CTF, TFIIA, TFIIB, TFIID, Pit-I, C/EBP, SRF (Mitchell, P. J. and Tijan, R. (1989) Science 245:371), which may be assayed in accordance with the invention for binding to the sequences of the invention.

In another embodiment of the invention, enhancer sequences of the present invention may be used to repress enhancer activity. For example, multiple copies of SCE1 may be introduced into a cell which exhibits SCE1 enhancer activity. These sequences may be either in the form of oligonucleotides or contained on a DNA vector separate from the vector containing the gene to be regulated. These DNA enhancer elements not in cis with the gene to be regulated compete for and reduce binding of transcription factors to the enhancer element and thereby repress enhancer activity. By “multiple copies” is meant to include a multiple of DNA sequences which is significantly high to cause competition and reduce binding of the transcription factors to the enhancer element and thereby repress enhancer activity. The number of enhancer sequences required to be provided in trans may be determined by transfecting a certain number of sequences into a cell containing a reporter gene under the control of the enhancer. The number of sequences is sufficient when the activity of the reporter gene is significantly repressed.

In another embodiment of the invention, a method of selecting agents which regulate SCE1 and/or SCE2 enhancer activity is provided. The method includes inserting the SCE1 and/or SCE2 nucleic acid sequence and a reporter gene into a vector and inserting the vector-into a cell. The cell is then exposed to the agent suspected of affecting SCE1 and/or SCE2 enhancer activity and the transcription efficiency is measured by the activity of the reporter gene. The activity can then be compared to the activity of the reporter gene in cells unexposed to the agent in question. The compounds and experimental conditions presently known to modulate myelin gene expression in Schwann cells and oligodendrocytes include progestins, that potentiate myelination both in vivo and in vitro, and electrical stimulation, that modulates the level of MBP expression in cultured Schwann cells (Koenig. et al., 1995; Stevens et al., 1998). It also is well established that elevation of cAMP levels in cultured Schwann cells results in the up-regulation of myelin gene expression (Lemke and Chao, 1988; Morgan et al., 1991). More recently, a role for myelin associated glycoprotein (MAG) in bi-directional transduction of axon-Schwann cell signals has been proposed based on the observation that homozygous MAG null animals express both axonal and myelin anomalies (Li et al., 1994; Yin et al., 1998). However, a putative downstream effector of MAG, Fyn tyrosine kinase, does not appear to be required for PNS myelination (Fujita et al., 1998; Osterhout et al., 1999).

Although various embodiments of the invention are disclosed herein, many adaptations and modifications may be made within the scope of the invention in accordance with the common general knowledge of those skilled in this art Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLE 1

Reporter Gene Constructs

Reporter gene constructs were constructed to localize cis-regulatory sequences that control MBP expression in Schwann cells. A 15 kb BamH I MBP genomic fragment, containing approximately 12.0 kb of MBP 5′ flanking sequence, was obtained by screening a lambda DASH-129 mouse genomic library (J. Rossant, Mount Sinai, Toronto). This sequence was subcloned in the BamHI site of pSK (13 clone). A Sac II (−9.0 kb)/Xba I (−3.1 kb) fragment from B clone was inserted into the reeve sites in clone pm12 (3.1 kb MBP 5′ flanking sequence in Xba I/Xma I sites of pSK- (Foran and Peterson, 1992)) to generate the −9.0 kb MBP promoter (clone 8). B clone was digested with Sac II followed by intramolecular ligation of the Sac II ends to generate the −12.0 kb (BamH I) to −9.0 kb (Sac II) MBP 5′ flanking sequence in pSK (B subclone 1A) (all vectors other than pm12 from Stratagene, La Jolla, Calif.; restriction enzymes from New England BioLabs, Mississauga, ON).

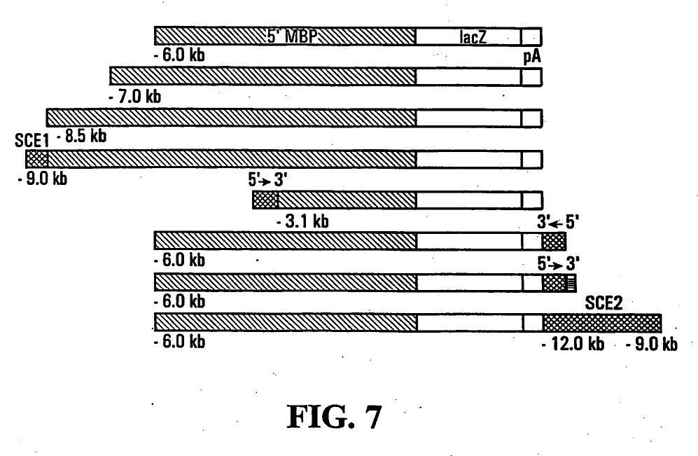

To generate reporter constructs, d10 lacZ (Foran and Peterson, 1992) was released from a pUC18 subclone with Sal I and cloned in the Sal I site of clone 8 (3′ to the −9 kb MBP promoter). This clone contained a double insert of lacZ and the second insert was released by BamH I digestion followed by intramolecular ligation (clone 5). As shown in FIG. 7, constructs containing 9.0 kb (Sac II), 8.5 kb (Nae I), 7.0 (Sph I), or 6.0 kb (Kpn I) of MBP promoter were obtained by restriction digestion of clone 5 and agarose (Boehringer Mannheim, Laval, PQ) gel purification (0.5%/TAE). A Kpn I/BamH I fragment of clone 5 (containing −6.0 kb MBP-lacZ) was cloned into the respective sites in B subclone 1A to generate a clone containing the −12.0 to −9.0 5′ MBP fragment at the 3′ end of lacZ in 5′ to 3′ orientation. This clone was digested with Kpn I/Sac II and the construct was gel purified as described above.

Two constructs were used to test the position and orientation independence of SCE1. SCE1 (0.6 kb; Sac II/Sac I) was isolated from clone 5 by Sac I digestion and cloned in the Sac I site of pSK+ (clone 6). −6.0 kb MBP-lacZ was isolated from clone S (Kpn I/BamH 1) and cloned in the same sites in clone 6 to generate −6.0 MBP-lacZ-5(SCE1)₃′ (FIG. 7). The construct was obtained by linearizing with Kpn I. To test the reverse orientation, SCE1 was isolated from clone 5 (Sac I) and cloned in the Sac I site of pBS (clone 7). −6.0 MBP-lacZ was isolated from clone 5 with Kpn I and cloned in the Kpn I site of clone 7 to generate −6.0 MBP-lacZ-3′(SCE1)5′ (FIG. 7). The construct was released with Sph I/Sac II for pronuclear injection.

A clone containing SCE1 and −3.1 kb MBP-lacZ also was generated. −3.1 kb MBP-lacZ was isolated from clone pm12 with Xba I and cloned in the Xba I site of clone 7, resulting in a clone containing 5(SCE1)3′- −3.1 kb MBP-lacZ). The construct was released with Sac II/Sph I.

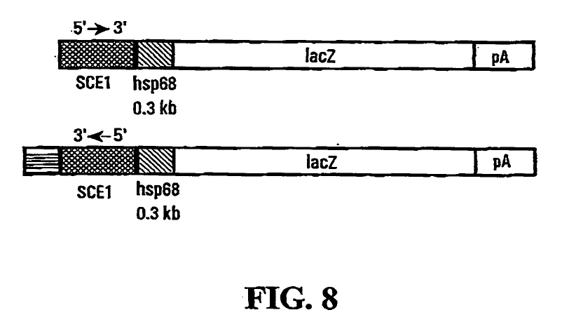

To generate SCE1-hsp-lacZ constructs (FIG. 8), the minimal 0.3 kb hsp68 promoter (Hind III/Nco I) ligated to lacZ (clone p610ZA; R Kothary, University of Ottawa) was used. The 0.6 kb SCE1 (Sac I/Sac I) was blunted (Klenow (Boehringer Mannheim, Laval, PQ)) and inserted in the EcoR V site of pKS+. In clone KS-SCE 8A, the 5′ end of the enhancer is closest to the Sac II site of the pKS+multiple cloning site (and the 3′ end closest to the Kpn I site). 03 kb hsp68-lacZ was isolated from clone p610ZA (Hind III/Kpn I) and cloned in the same sites in KS-SCE 8A to gene SCE-hsp 2G (5(SCE1)₃′-hsp68-lacZ in pKS+). The construct was released by Sma I digestion and purified as above. To generate a construct having the SCE1 in 3′ to 5′ orientation, the 0.6 kb SCE1 (Sac II/Sac I) was cloned in the Sac II/Sac I sites of pSK+(clone SK-S CE). 0.3 kb hsp68-lacZ was isolated from p610ZA (Hind III/Kpn I) and cloned in the same sites in SK-SCE (clone SCE-hsp 1B: 3′(SCE1)₅′-hsp68-lacZ). This construct was linearized with Kpn I.

### EXAMPLE 2

Transgenic Mice

Transgenic mice were derived by injection of DNA into the pronuclei of B6C3F2 zygotes as previously described (Foran and Peterson, 1992). Injected zygotes were transplanted into the oviducts of B6C3F1 females rendered pseudopregnant by mating with vasectomized males. Litters were delivered either spontaneously or by cesarean section 18 days later. Primary transgenic mice and mice derived from established lines were investigated for transgene expression (Table 1).

### EXAMPLE 3

Histochemical Detection of β-Galactosidase Activity

Mice were anesthetized with avertin intraperitoneally and perfused transcardially with 0.5% paraformaldehyde, 2.5% glutaraldehyde in 0.1 M phosphate buffer (pH 7.4) all at 4° C. Tissues to be analyzed were recovered and postfixed for an additional hour. Samples were then rinsed in 0.1 M phosphate buffer and incubated as whole mounts at 37° C. for various times ranging from less than one hour to overnight in stain consisting of 2 mM MgCl₂, 5 mM K₃Fe(CN)₆, 5 mM K₄Fe(CN)₆ and 0.4 mg/ml Bluo-gal (Gibco/BRL, Burlington, Ontario). In some preparations, detergents sodium deoxycholate and NP-40 were added to the stain at 0.01% and 0.03% respectively to permeabilize the tissue thus assisting the penetration of β-galactosidase substrate. Following the whole mount histochemical reaction, some samples were processed for plastic embedding. Typically, tissue was osmicated prior to dehydration and embedding in Epon. Following polymerization, the blocks were sectioned at 1 μm, and the sections were mounted on slides and viewed either directly or after staining with toluidine blue. Additional tissue was cryoprotected by immersion in 30% sucrose prior to freezing and 12 μm cryostat sections were made and subsequently incubated in stain containing 0.8 mg/ml X-gal (Gibco/BRL, Burlington, Ontario). To investigate prenatal expression, we applied a histochemical technique capable of detecting low-level β-galactosidase activity in sections. Fetuses were recovered and immersion fixed for 1 hour in the same aldehyde mix at 4° C. To cryoprotect, they were then incubated at 4° C. in 30% sucrose overnight prior to freezing in isopentane pre-cooled in liquid nitrogen. Cryostat sections, 12 μm thick, were picked up on slides and dried for 30 min at room temperature and 20 min at 37° C. Sections were then post-fixed by immersion in 4% formalin and 7.5% sucrose in 0.1 M phosphate buffer for 20 min at 4° C., washed for 5 min in buffer alone 3 times, then transferred to β-galactosidase stain (X-gal at 0.8 mg/ml) and incubated at 37° C. overnight. Slides were then cover-slipped using 10% glycerol as the mounting medium.

### EXAMPLE 4

Identification of Mouse SCE1

Using the reporter gene constructs described above, the expression conferred to MBP was analyzed in transgenic mice. In the initial constructs evaluated, lacZ was promoted by approximately 6, 7, 8.5 or 9 kb of MBP 5′ flanking sequence (FIG. 7). Constructs containing flanking sequences extending to −8.5 kb expressed only in oligodendrocytes while the construct regulated by the −9.0 kb sequence also expressed in Schwann cells (Table 1) indicating that one or more Schwann cell targeting elements are located in the sequence between −9 and −8.5 kb.

To assess whether this distal sequence had functions characteristic of an enhancer, constructs were generated in which the slightly longer −9 to −8.4 kb sequence was ligated, in both orientations, to the 3′ end of the lacZ reporter promoted by 6 kb of MBP 5′ flanking sequence (FIG. 7). In another construct, it was ligated immediately upstream of 3.1 kb of MBP 5′ flanking sequence driving lacZ. Consistent with classic enhancer function, robust expression was observed in the PNS of multiple lines bearing all three constructs (Table 1). The Schwann cell targeting conferred by this 0.6 kb MBP sequence was assigned the interim designation, Schwann Cell Enhancer 1 (SCE1).

### EXAMPLE 5

Identification of Mouse SCE2

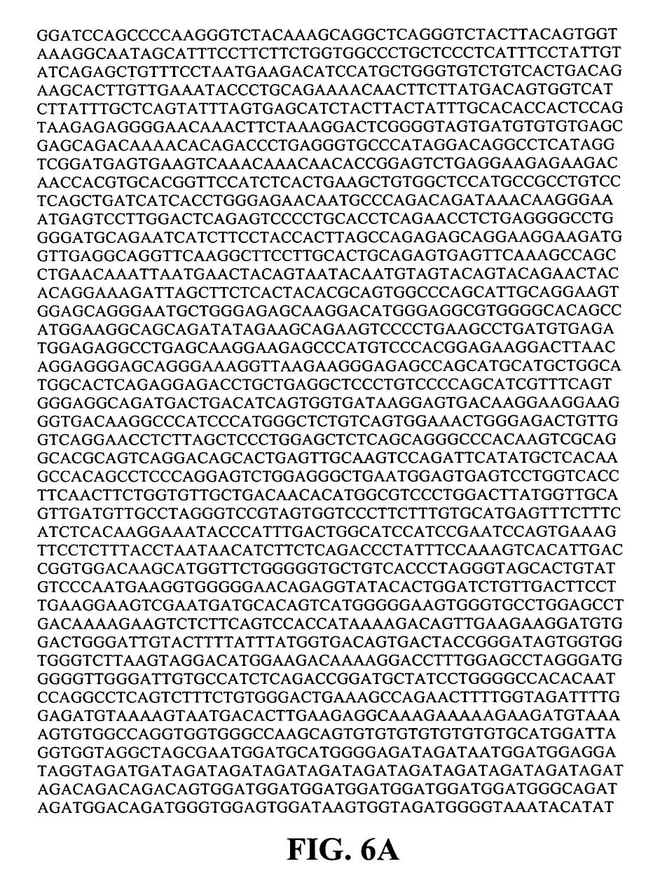

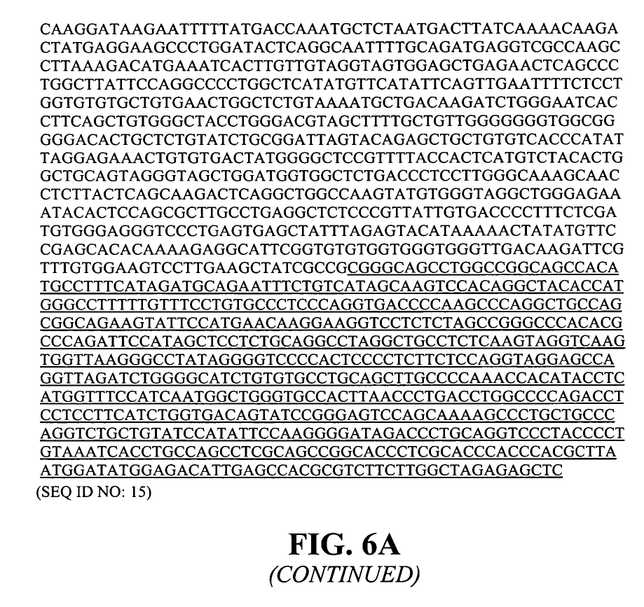

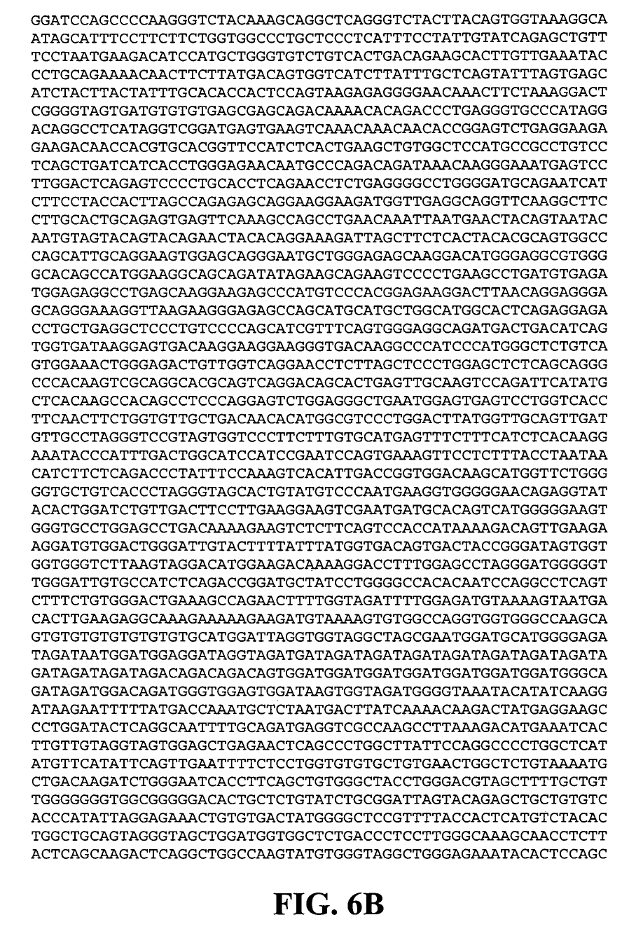

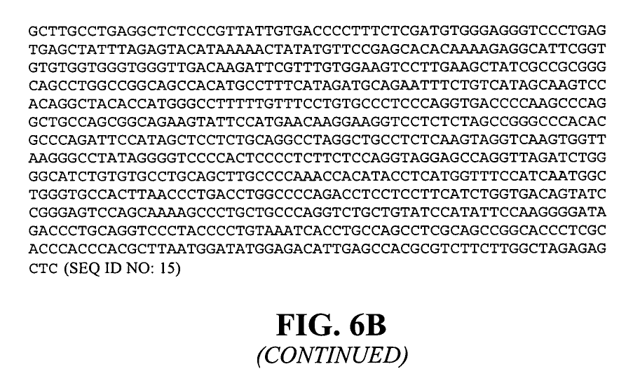

To search for further sequences capable of conferring Schwann cell expression, the 3 kb sequence immediately upstream of SCE1 (−12.0 to −9.0) was ligated to the 3′ end of the lac Z reporter gene, driven by 6 kb of MBP 5′ flanking sequence (FIG. 7). So far, one transgenic mouse that failed to transmit the transgene has been analyzed. Although expression of the transgene was mosaic, those Schwann cells that labeled did so intensely (Table 1). As the MBP sequence in this construct was ligated 3′ of lacZ, its provisional targeting ability also appeared to be mediated through enhancer activity and it was assigned the interim designation, Schwann Cell Enhancer 2 (SCE2). The sequence extending from −12 kb to −8.4 kb (including both SCE1 and SCE2) has been deposited to GenBank (accession number AF277397) (FIG. 6).

### EXAMPLE 6

Heterologous Promoter Constructs

As shown in FIG. 8, the −9.0 to −8.4 kb sequence was ligated, in both orientations, 5′ of a heterologous promoter (0.3 kb hsp68) driving the lacZ reporter gene to determine whether the Schwann cell targeting activity of SCE1 is independent of elements contained within the proximal MBP promoter. Transgenic mice bearing this SCE1-hsp68-lacZ construct were derived and seven of ten independent transgenic lines, including those bearing SCE1 in either orientation, expressed β-galactosidase in myelin forming Schwann cells (Table 1). These constructs were expressed at high levels in the myelinating Schwann cells of multiple lines, indicating that SCE1 contains robust Schwann cell enhancer activity that is sufficient to target Schwann cell expression.

### EXAMPLE 7

Identification of Homologous Human SCE1 Sequence

Using sequence comparison algorithms with human and mouse MBP 5′ flanking sequence, a homologous and potentially enhancer-bearing human sequence was identified at approximately −16 kb in the human locus (FIGS. 1, 2, and 4). A 1.1 kb human sequence, derived from an M13 mp 18 library containing 2 kb fragments of human MBP genomic DNA, containing the 0.6 kb region of human/mouse homology was incorporated into a minimally promoted lacZ reporter construct Subclone 532 (containing the human MBP 5′ flanking sequence from −15.5 Kb to −13.5 Kb) was digested with EcoRI and HindIII and ligated into the polylinker of pKS+. This construct was-digested with XbaI and BssSI, blunted with klenow, and a fragment containing 1022 bp of human MBP 5′ flanking sequence (from −15.5 Kb to −14.5 Kb) (“module 4”) was gel extracted. This fragment was ligated in a 5′-3′ orientation into the Pmel site of a modified version of a hsp70-lacZ containing vector described previously. A human MBP module 4-hsp-lacZ fragment was isolated from the resultant vector by digestion with AscI and NotI and inserted into a modified version of the PMP8SKB HPRT targeting vector described by Bronson et al (1996). This vector was linearized by digestion with SalI and 40 ug of plasmid DNA was used for ES cell transfection. When introduced into the genome of mice, the human MBP module 4 demonstrated strong Schwann cell enhancer activity.

### EXAMPLE 8

SCE1 Directs Endogenous MBP Expression Program

To determine how closely the expression conferred by SCE1 tracks the expression phenotype of the endogenous MBP gene, prenatal and postnatal transgenic mice bearing SCE1-containing constructs were analyzed. The endogenous MBP locus is expressed at low but detectable levels in the developing PNS of prenatal mice (Bachnou et al., ISN Satellite and UConn-Kroc Symposium, Mystic, 1997). Transgenic mice bearing SCE1-hsp68-lacZ constructs similarly express low but detectable levels of β-galactosidase activity in fetal peripheral nerves from E14 through birth. Also reflecting the endogenous expression program, high-level expression of β-galactosidase appeared in Schwann cells coincident with myelin formation when accumulation of endogenous MBP mRNA markedly increases. As predicted by the temporally discordant myelination programs in dorsal and ventral spinal roots of mice (Baron et al., 1994), ventral roots began to label intensely one to two days prior to dorsal roots. During the first postnatal week of mouse development, the number of myelinating Schwann cells greatly increases (Bray et al., 1977) and the apparent level of β-galactosidase accumulation, assessed by the rapidity and intensity of histochemical labeling, increased in parallel. As similar expression programs were observed for SCE1 containing constructs promoted either by hsp68 or proximal MBP sequence, it appears that this short enhancer sequence is capable of directing the major features of the endogenous MBP expression program during Schwann cell maturation.

### EXAMPLE 9

Differential Expression of SCE1

As mice bearing the 9.0 kb promoted construct matured, there was a modest decline in β-galactosidase labeling intensity throughout their mixed nerves (lines 17 and 32) and in the mosaic line 24, expression ceased. Surprisingly and consistently, in mice over 3 months of age from line 17 and 32, dorsal roots continued to label but a further precipitous decline in transgene expression was observed in ventral roots. In line 17 mice, expression appeared to be shut off while in line 32, ventral root down regulation was not absolute but labeling intensity was clearly weaker than that observed in dorsal roots. A similarly dramatic difference in dorsal and ventral root labeling intensity was observed in one line of SCE1-hsp68-lacZ mice (also designated 17). These combined observations suggest a novel level of heterogeneity amongst Schwann cells that coincides with the modality (sensory versus motor) of the innervating axon. Although a striking finding in the multiple lines where it was observed, this phenomenon was not encountered in all SCE1 bearing lines. Notably, both dorsal and ventral roots in mice from the SCE1-hsp68-lac Z lines 49 and 54 continued to label at an apparently similar level throughout maturity.

### EXAMPLE 10

Analysis of SCE1 and SCE2 Sub-Regions and their Effects on SCE1 and SCE2 Enhancer Activity

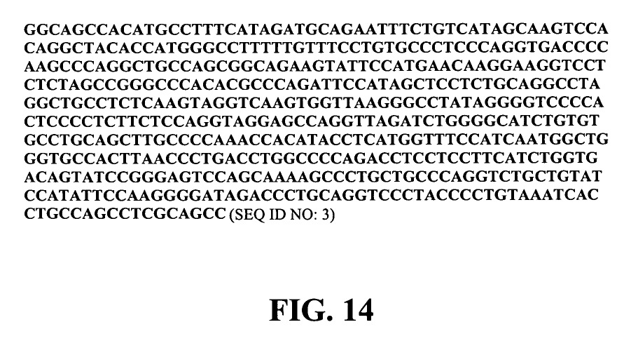

A 510 bp SCE1 sub-sequence defined by 5′ NaeI and 3′ NaeI restriction sites (FIG. 14) leads to expression in myeling bearing Schwann cells, albeit at weaker levels than that observed using full length SCE1. The construct demonstrating this Schwann cell enhancer activity consisted of a promoter contained within the 3.1 Kb of mouse MBP 5′ flanking sequence (defined on its 5′ end by an XbaI site), ligated to the lac Z reporter followed by the above SCE1 sequence.

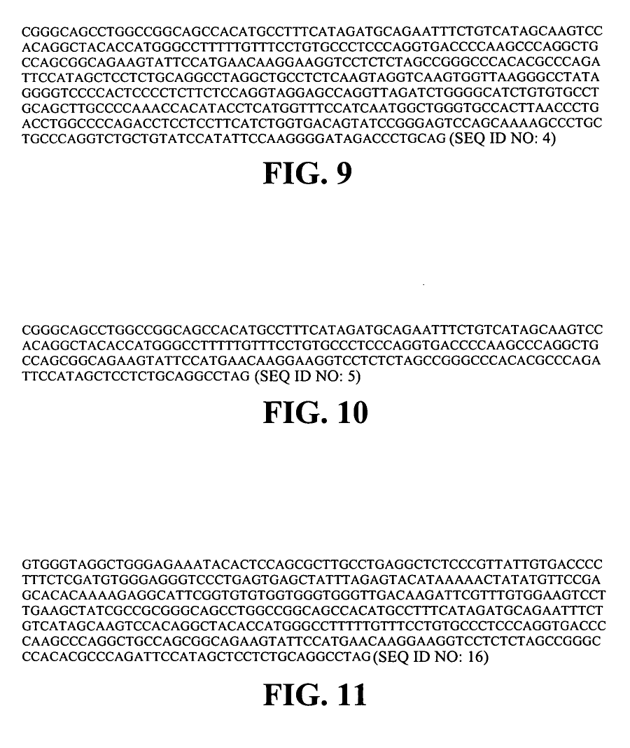

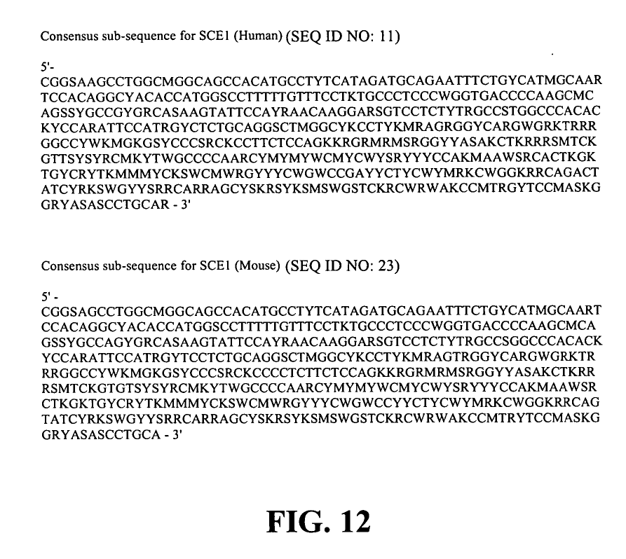

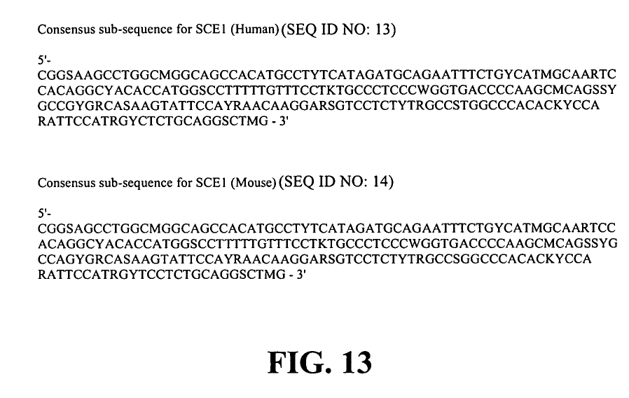

A 488 bp SCE1 sub-sequence defined by 5′ SacII and 3′ Sbf1 restriction sites (FIG. 9) leads to expression in myeling bearing Schwann cells, albeit at weaker levels than that observed using full length SCE1.

A shorter, 213 bp SCE1 sub-sequence defined by SacII and AvrII restriction sites (FIG. 10) leads to strong expression in myelin bearing Schwann cells. Moreover, its Schwann cell targeting activity is conferred with a heterologous 300 bp hsp “minimal” promoter, i.e., in the absence of further MBP sequences. The construct demonstrating this Schwann cell enhancer activity consisted of the above sequence ligated to the proximal 267 bp of 5′ flanking sequence from the mouse hpp 68 gene* followed by the lacZ reporter.

*mouse hsp promoter sequence
GTGAAGACTCCTTAAAGGCGCAGGGCGGCGAGCAGGTCACCAGACGCTGACA
GCTACTCAGAACCAAATCTGGTTCCATC
CAGAGACAAGCGAAGACAAGAGAAGCAGACCGAGCGGCGCGTTCCCGATCCT
CGGCCAGGACCAGCCTTCCCCAGAGCAT
CCCTGCCGGGACGAACCTTCCCAGGAGCATCCCTGCCGGGAGCGGAACTTTCC
CCGGAGCATCCAGCCGCGGACGCAGCC
TTCCAGAAGCACAGCGCGGCGCCATGG

A construct bearing the 213 bp sequence noted above, plus 203 bp of contiguous 5′ mouse sequence from SCE2 (a total of 416 bps; FIG. 11) is again a strong enhancer conferring high levels of expression to myelin bearing Schwann cells throughout both the juvenile stage and mature life of mice. The construct demonstrating this Schwann cell enhancer activity consisted of the sequence of FIG. 11 ligated to the proximal 267 bp of 5′ flanking sequence from the mouse hpp 68 gene (*as above) followed by the lacZ reporter.

The above results suggest that different regions of SCE1 confer different types and levels of modulation of transcriptional activity. For example, the higher level of enhancer activity observed with the above noted 213 bp sequence relative to the larger 510 bp and 486 bp sequences, demonstrates the presence of regions in the 510 bp and 486 bp sequences which may confer some repression of transcriptional activity when present in a certain context. These results thus further demonstrate that the nucleic acids of the invention can provide a variable and fine control for the modulation of transcriptional activity, both in the type of modulation (enhancement or repression) and the degree, which can thus be tailored to meet the requirements of various specific applications.

TABLE 1

| | Transgenic | | |
|---|---|---|---|
| | line(#) or | Postnatal expression | |
| Construct | 1°* | CNS | PNS |
| −6.0 kb MBP + lacZ | #2 | + | − |
| | #5 | + | − |
| −7.0 kb MBP + lacZ | #11 | + | − |
| | #18 | + | − |
| | #26 | + | − |
| −8.5 kb MBP + lacZ | #4 | + | − |
| −9.0 kb MBP + lacZ | #7 | − | − |
| | #17 | +** | + |
| | #24 | + | + |
| | #32 | + | + |
| SCE1 (5′→3′) + −3.1 kb MBP + | #12 | + | + |
| lacZ | #28 | + | + |
| | #32 | − | − |
| −6.0 kb MBP + lacZ + SCE1 (3′→ | #9 | + | + |
| 5′) | #10 | + | + |
| | #16 | + | + |
| −6.0 kb MBP + lacZ + SCE1 (5′→ | #7 | + | + |
| 3′) + pSK | | | |
| −6.0 kb MBP + lacZ + SCE2 (5′→ | 1° | + | + |
| 3′) | | | |
| SCE1 (5′→3′) + hsp + lacZ | #17 | ** | + |
| | #18 | − | + |
| | #20 | − | − |
| | #21 | − | − |
| | #40 | ** | + |
| | #42 | ** | + |
| | #46 | − | − |
| | #49 | − | + |
| | #54 | ** | + |
| SCE1 (3′→5′) + hsp + lacZ + pSK | 1° | ** | + |
| −6.0 kb MBP + lacZ + δKrox- | #4 | + | + |
| 20-SCE1 (5′→3′) | | | |
| transgenic mice used for the functional analysis of the MBP promoter. The designations + (indicates reporter gene expression) and − (no expression) were made based on the β-galactosidase histochemical assay as described. Designations were made based on comprehensive analysis | | | |
| # that included samples from the period of maximal myelin gene expression in early post-natal development (P3-P21). Constructs designated (+) were defined by obvious staining that contrasted to the failure to stain in littermates processed during the same experiment. | | | |

*1° indicates that a primary transgenic mouse, rather than mice from a transgenic line, was analyzed for a given construct.
**Lines known to show unique patterns of expression outside the PNS attributed to enhancer trapping at the site of transgene insertion.

### REFERENCES

All publications, including patent applications referred to herein and including the following, are hereby incorporated by reference (no admission is hereby made with respect to whether such references constitute prior art in any jurisdiction):
Aguayo A J, Charron L, Bray G M (1976a) Potential of Schwann cells from unmyelinated nerves to produce myelin: a quantitative ultrastuctural and radiographic study. J Neurocytol 5:565-573.
Aguayo A J, Epps J, Charron L, Bray G M (1976b) Multipotentiality of Schwann cells in cross-anastomosed and grafted myelinated and unmyelinated nerves: quantitative microscopy and radioautography. Brain Res 104:1-20.
Baron P, Shy M, Kamholz J, Scarlato G, Pleasure D (1994) Expression of P0 protein mRNA along rat sciatic nerve during development. Brain Res Dev Brain Res 83:285-288.
Bermingham J R, Jr., Scherer S S, O'Connell S, Arroyo E, Kalla K A, Powell F L, Rosenfeld M G (1996) Tst-1/Oct-6/SCIP regulates a unique step in peripheral myelination and is required for normal respiration. Genes Dev 10:1751-1762.
Berthold C H (1978) Morphology of normal peripheral axons. In: Physiology and pathobiology of axons (Waxman S G, ed), pp 3-63. New York: Raven Press.
Bowe C M, Kocsis J D, Waxman S G (1985) Differences between mammalian ventral and dorsal spinal roots in response to blockade of potassium channels during maturation Proc R Soc Lond B Biol Sci 224:355-366.
Bray G M, Perkins S, Peterson A C, Aguayo A J (1977) Schwann cell multiplication deficit in nerve roots of newborn dystrophic mice. A radioautographic and ultrastructural study. J Neurol Sci 32:203-212.
Bronson S K, Plaehn E G, Kluckman K D, Hagaman J R, Maeda N, Smithies O (1996) Single-copy transgenic mice with chosen-site integration [see comments]. Proc Natl Acad Sci USA 93:9067-9072.
Chavrier P. Vesque C, Galliot B, Vigneron M, Dolle P, Duboule D, Charnay P (1990) The segment-specific gene Krox-20 encodes a transcription factor with binding sites in the promoter region of the Hox-1.4 gene. Embo J 9:1209-1218.
Farhadi H F, Forghani R, Peterson A C (1999) MBP-lacZ transgene expression in juvenile and adult Trembler-J mice. Ann N Y Acad Sci 883:538-539.
Foran D R, Peterson A C (1992) Myelin acquisition in the central nervous system of the mouse revealed by an MBP-Lac Z transgene. J Neurosci 12:48904897.
Forghani R, Nesbitt J, Snipes J, Shooter E M, Peterson A (1999) Preparation of nuclear extracts from myelinating Schwann cells. J Neurosci Methods 89:129-132.
Fujita N, Kemper A, Dupree J, Nakayasu H, Bartsch U, Schachner M, Maeda N, Suzuki K, Popko B (1998) The cytoplasmic domain of the large myelin-associated glycoprotein isoform is needed for proper CNS but not peripheral nervous system myelination. J Neurosci 18:1970-1978.
Goujet-Zalc C, Babinet C, Monge M, Timsit S, Cabon F, Gansmuller A, Miura M, Sanchez M, Pournin S, Mikoshlba K, et al. (1993) The proximal region of the MBP gene promoter is sufficient to induce oligodendroglial-specific expression in transgenic mice. Eur J Neurosci 5:624-632.
Gow A, Friedrich V L, Jr., Lazzarini R A (1992) Myelin basic protein gene contains separate enhancers for oligodendrocyte and Schwann cell expression. J Cell Biol 119:605-616.
Gravel M, Di Polo A, Valera P B, Braun P E (1998) Four-kilobase sequence of the mouse CNP gene directs spatial and temporal expression of lacZ in transgenic mice. J Neurosci Res 53:393404.
Gupta S K, Poduslo J F, Mezei C (1988) Temporal changes in PO and MBP gene expression after crush-injury of the adult peripheral nerve. Brain Res 464:133-141.
He X, Gerrero R, Simmons D M, Park R E, Lin C J, Swanson L W, Rosenfeld M G (1991) Tst-1, a member of the POU domain gene family, binds the promoter of the gene encoding the cell surface adhesion molecule P0. Mol Cell Biol 11:1739-1744.
Heinemeyer T, Wingender E, Reuter I, Hermjakob H, Kel A E, Kel O V, Ignatieva E V, Ananko E A, Podkolodnaya O A, Kolpakov F A, Podkolodny N L, Kolchanov N A (1998) Databases on transcriptional regulation: TRANSFAC, TRRD and COMPEL. Nucleic Acids Res 26:362-367.
Jaegle M, Mandemakers W, Broos L, Zwart R, Karis A, Visser P, Grosveld F, Meijer D (1996) The POU factor Oct-6 and Schwann cell differentiation. Science 273:507-510.
Koenig H L, Schumacher M, Ferzaz B, Thi A N, Ressouches A, Guennoun R, Jung-Testas I, Robel P, Akwa Y, Baulieu E E (1995) Progesterone synthesis and myelin formation by Schwann cells. Science 268:1500-1503.
Lamperth L, Manuelidis L, Webster H D (1990) P0 glycoprotein mRNA distribution in myelin-forming Schwann cells of the developing rat trigeminal ganglion. J Neurocytol 19:756-764.
LeBlanc A C, Poduslo J F (1990) Axonal modulation of myelin gene expression in the peripheral nerve. J Neurosci Res 26:317-326.
Lemke G, Chao M (1988) Axons regulate Schwann cell expression of the major myelin and NGF receptor genes. Development 102:499-504.
Li C, Tropak M B, Gerlai R, Clapoff S, Abramow-Newerly W, Trapp B, Peterson A, Roder J (1994) Myelination in the absence of myelin-associated glycoprotein. Nature 369:747-750.
Martini R, Schachner M, Brushart T M (1994) The L2/HNK-1 carbohydrate is preferentially expressed by previously motor axon-associated Schwann cells in reinnervated peripheral nerves. J Neurosci 14:7180-7191.
Martini R, Zielasek J, Toyka K V, Giese K P, Schachner M (1995) Protein zero (P0)-deficient mice show myelin degeneration in peripheral nerves characteristic of inherited human neuropathies. Nat Genet 11:281-286.
Messing A, Behringer R R, Hammang J P, Palmiter R D, Brinster R L, Lemke G (1992) P0 promoter directs expression of reporter and toxin genes to Schwann cells of transgenic mice. Neuron 8:507-520.
Mirsky R, Jessen K R (1996) Schwann cell development, differentiation and myelination. Curr Opin Neurobiol 6:89-96.
Miskimins R, Knapp L, Dewey M J, Zhang X (1992) Cell and tissue-specific expression of a heterologous gene under control of the myelin basic protein gene promoter in transgenic mice. Brain Res Dev Brain Res 65:217-221.
Monuki E S, Weinmaster G, Kuhn R, Lemke G (1989) SCIP: a glial POU domain gene regulated by cyclic AMP. Neuron 3:783-793.
Monuki E S, Kuhn R, Weinmaster G, Trapp B D, Lemke G (1990) Expression and activity of the POU transcription factor SCIP. Science 249:1300-1303.
Morgan L, Jessen K R, Mirsky R (1991) The effects of cAMP on differentiation of cultured Schwann cells: progression from an early phenotype (04+) to a myelin phenotype (P0+, GFAP-, N-CAM-, NGF-receptor-) depends on growth inhibition. J Cell Biol 112:457-467.
Murphy P, Topilko P, Schneider-Maunoury S, Seitanidou T, Baron-Van Evercooren A, Charnay P (1996) The regulation of Krox-20 expression reveals important steps in the control of peripheral glial cell development. Development 122:2847-2857.
Osterhout D J, Wolven A, Wolf R M, Resh M D, Chao M V (1999) Morphological differentiation of oligodendrocytes requires activation of Fyn tyrosine kinase. J Cell Biol 145:1209-1218.
Peterson A C, Bray G M (1984) Normal basal laminas are realized on dystrophic Schwann cells in dystrophic in equilibrium shiverer chimera nerves. J Cell Biol 99:1831-1837.
Sham M H, Vesque C, Nonchev S, Marshall H, Frain M, Gupta R D, Whiting J, Wilkinson D, Chamay P, Krumlauf R (1993) The zinc finger gene Krox20 regulates HoxB2 (Hox2.8) during hindbrain segmentation. Cell 72:183-196.
Snipes G J, Suter U, Welcher A A, Shooter E M (1992) Characterization of a novel peripheral nervous system myelin protein (PMP-22/SR13). J Cell Biol 117:225-238.
Stahl N, Harry J, Popko B (1990) Quantitative analysis of myelin protein gene expression during development in the rat sciatic nerve. Brain Res Mol Brain Res 8:209-212.
Stankoff B, Demerens C, Goujet-Zalc C, Monge M, Peyron F, Mikoshiba K, Zalc B, Lubetzzi C (1996) Transcription of myelin basic protein promoted by regulatory elements in the proximal 5′ sequence requires myelinogenesis. Mult Scler 2:125-132.
Stevens B, Tanner S, Fields R D (1998) Control of myelination by specific patterns of neural impulses. J Neurosci 18:9303-9311.
Topilko P, Schneider-Maunoury S, Levi G. Baron-Van Evercooren A, Chennoufi A B, Seitanidou T, Babinet C, Charnay P (1994) Krox-20 controls myelination in the peripheral nervous system. Nature 371:796-799.
Trapp B D, Hauer P, Lemke G (1988) Axonal regulation of myelin protein mRNA levels in actively myelinating Schwann cells. J Neurosci 8:3515-3521.
Weinstein D E, Burrola P G, Lemke G (1995) Premature Schwann cell differentiation and hypermyelination in mice expressing a targeted antagonist of the POU transcription factor SCIP. Mol Cell Neurosci 6:212-229.
Wrabetz L, Feltri M L, Quattrini A, Imperiale D, Previtali S, D'Antonio M, Martini R, Yin X, Trapp B D, Zhou L, Chiu S Y, Messing A (2000) P(O) glycoprotein overexpression causes congenital hypomyelination of peripheral nerves. J Cell Biol 148:1021-1034.
Yin X Crawford T O, Griffin J W, Tu P, Lee V M, Li C, Roder J, Trapp B D (1998) Myelin-associated glycoprotein is a myelin signal that modulates the caliber of myelinated axons. J Neurosci 18:1953-1962.
Yuh C H, Bolouri H, Davidson E H (1998) Genomic cis-regulatory logic: experimental and computational analysis of a sea urchin gene [see comments]. Science 279:1896-1902.
Zorick T S, Lenke G (1996) Schwann cell differentiation. Curr Opin Cell Biol 8:870-876.

1. An isolated nucleic acid molecule comprising a nucleic acid sequence effective to modulate transcription of operably-linked sequences in Schwann cells.

2. The isolated nucleic acid molecule of claim 1, wherein the nucleic acid sequence comprises:
(SEQ ID NO: 2)
CGGCAGCCTGGCCGGCAGCCACATGCCTTTCATAGATGCAGAATTTC
TGTCATAGCAAGTCCACAGGCTACACCATGGGCCTTTTTGTTTCCTGT
GCCCTCCCAGGTGACCCCAAGCCCAGGCTGCCAGCGGCAGAAGTATT
CCATGAACAAGGAAGGTCCTCTCTAGCCGGGCCCACACGCCCAGATT
CCATAGCTCCTCTGCAGGCCTAGGCTGCCTCTCAAGTAGGTCAAGTG
GTTAAGGGCCTATAGGGGTCCCCACTCCCCTCTTCTCCAGGTAGGAG
CCAGGTTAGATCTGGGGCATCTGTGTGCCTGCAGCTTGCCCCAAACC
ACATACCTCATGGTTTCCATCAATGGCTGGGTGCCACTTAACCCTGAC
CTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAGGTCCCTACCCCTGTAAATCACCTGCCAGCCTCGC
AGCCGGCACCCTCGCACCCACCCACGCTTAATGGATATGGAGACATT
GAGCCACGCGTCTTCTTGGCTAGAGAGCTCTGAGCAGCCTCTCCCGA
GC.

3. The isolated nucleic acid molecule of claim 1, wherein the nucleic acid sequence hybridizes under stringent conditions to the following sequence or its complement:
(SEQ ID NO: 2)
CGGCAGCCTGGCCGGCAGCCACATGCCTTTCATAGATGCAGAATTTC
TGTCATAGCAAGTCCACAGGCTACACCATGGGCCTTTTTGTTTCCTGT
GCCCTCCCAGGTGACCCCAAGCCCAGGCTGCCAGCGGCAGAAGTATT
CCATGAACAAGGAAGGTCCTCTCTAGCCGGGCCCACACGCCCAGATT
CCATAGCTCCTCTGCAGGCCTAGGCTGCCTCTCAAGTAGGTCAAGTG
GTTAAGGGCCTATAGGGGTCCCCACTCCCCTCTTCTCCAGGTAGGAG
CCAGGTTAGATCTGGGGCATCTGTGTGCCTGCAGCTTGCCCCAAACC
ACATACCTCATGGTTTCCATCAATGGCTGGGTGCCACTTAACCCTGAC
CTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAGGTCCCTACCCCTGTAAATCACCTGCCAGCCTCGC
AGCCGGCACCCTCGCACCCACCCACGCTTAATGGATATGGAGACATT
GAGCCACGCGTCTTCTTGGCTAGAGAGCTCTGAGCAGCCTCTCCCGA
GC.

4. The isolated nucleic acid molecule of claim 1, wherein the nucleic acid sequence is 95% identical to the following sequence when optimally aligned:
(SEQ ID NO: 2)
CGGCAGCCTGGCCGGCAGCCACATGCCTTTCATAGATGCAGAATTTC
TGTCATAGCAAGTCCACAGGCTACACCATGGGCCTTTTTGTTTCCTGT
GCCCTCCCAGGTGACCCCAAGCCCAGGCTGCCAGCGGCAGAAGTATT
CCATGAACAAGGAAGGTCCTCTCTAGCCGGGCCCACACGCCCAGATT
CCATAGCTCCTCTGCAGGCCTAGGCTGCCTCTCAAGTAGGTCAAGTG
GTTAAGGGCCTATAGGGGTCCCCACTCCCCTCTTCTCCAGGTAGGAG
CCAGGTTAGATCTGGGGCATCTGTGTGCCTGCAGCTTGCCCCAAACC
ACATACCTCATGGTTTCCATCAATGGCTGGGTGCCACTTAACCCTGAC
CTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAGGTCCCTACCCCTGTAAATCACCTGCCAGCCTCGC
AGCCGGCACCCTCGCACCCACCCACGCTTAATGGATATGGAGACATT
GAGCCACGCGTCTTCTTGGCTAGAGAGCTCTGAGCAGCCTCTCCCGA
GC.

5. The isolated nucleic acid molecule of claim 1, wherein the nucleic acid sequence is 90% identical to the following sequence when optimally aligned:
(SEQ ID NO: 2)
CGGCAGCCTGGCCGGCAGCCACATGCCTTTCATAGATGCAGAATTTC
TGTCATAGCAAGTCCACAGGCTACACCATGGGCCTTTTTGTTTCCTGT
GCCCTCCCAGGTGACCCCAAGCCCAGGCTGCCAGCGGCAGAAGTATT
CCATGAACAAGGAAGGTCCTCTCTAGCCGGGCCCACACGCCCAGATT
CCATAGCTCCTCTGCAGGCCTAGGCTGCCTCTCAAGTAGGTCAAGTG
GTTAAGGGCCTATAGGGGTCCCCACTCCCCTCTTCTCCAGGTAGGAG
CCAGGTTAGATCTGGGGCATCTGTGTGCCTGCAGCTTGCCCCAAACC
ACATACCTCATGGTTTCCATCAATGGCTGGGTGCCACTTAACCCTGAC
CTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAGGTCCCTACCCCTGTAAATCACCTGCCAGCCTCGC
AGCCGGCACCCTCGCACCCACCCACGCTTAATGGATATGGAGACATT
GAGCCACGCGTCTTCTTGGCTAGAGAGCTCTGAGCAGCCTCTCCCGA
GC.

6. The isolated nucleic acid molecule of claim 1, wherein the nucleic acid sequence is 80% identical to the following sequence when optimally aligned:
(SEQ ID NO: 2)
CGGCAGCCTGGCCGGCAGCCACATGCCTTTCATAGATGCAGAATTTC
TGTCATAGCAAGTCCACAGGCTACACCATGGGCCTTTTTGTTTCCTGT
GCCCTCCCAGGTGACCCCAAGCCCAGGCTGCCAGCGGCAGAAGTATT
CCATGAACAAGGAAGGTCCTCTCTAGCCGGGCCCACACGCCCAGATT
CCATAGCTCCTCTGCAGGCCTAGGCTGCCTCTCAAGTAGGTCAAGTG
GTTAAGGGCCTATAGGGGTCCCCACTCCCCTCTTCTCCAGGTAGGAG
CCAGGTTAGATCTGGGGCATCTGTGTGCCTGCAGCTTGCCCCAAACC
ACATACCTCATGGTTTCCATCAATGGCTGGGTGCCACTTAACCCTGAC
CTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAGGTCCCTACCCCTGTAAATCACCTGCCAGCCTCGC
AGCCGGCACCCTCGCACCCACCCACGCTTAATGGATATGGAGACATT
GAGCCACGCGTCTTCTTGGCTAGAGAGCTCTGAGCAGCCTCTCCCGA
GC.

7. The isolated nucleic acid molecule of claim 1, wherein the nucleic acid sequence comprises:
(SEQ ID NO: 3)
GGCAGCCACATGCCTTTCATAGATGCAGAATTTCTGTCATAGCAAGT
CCACAGGCTACACCATGGGCCTTTTTGTTTCCTGTGCCCTCCCAGGTG
ACCCCAAGCCCAGGCTGCCAGCGGCAGAAGTATTCCATGAACAAGG
AAGGTCCTCTCTAGCCGGGCCCACACGCCCAGATTCCATAGCTCCTC
TGCAGGCCTAGGCTGCCTCTCAAGTAGGTCAAGTGGTTAAGGGCCTA
TAGGGGTCCCCACTCCCCTCTTCTCCAGGTAGGAGCCAGGTTAGATC
TGGGGCATCTGTGTGCCTGCAGCTTGCCCCAAACCACATACCTCATG
GTTTCCATCAATGGCTGGGTGCCACTTAACCCTGACCTGGCCCCAGA
CCTCCTCCTTCATCTGGTGACAGTATCCGGGAGTCCAGCAAAAGCCC
TGCTGCCCAGGTCTGCTGTATCCATATTCCAAGGGGATAGACCCTGC
AGGTCCCTACCCCTGTAAATCACCTGCCAGCCTCGCAGCC.

8. The isolated nucleic acid molecule of claim 1, wherein the nucleic acid sequence hybridizes under stringent conditions to the following sequence or its complement:
(SEQ ID NO: 3)
GGCAGCCACATGCCTTTCATAGATGCAGAATTTCTGTCATAGCAAGT
CCACAGGCTACACCATGGGCCTTTTTGTTTCCTGTGCCCTCCCAGGTG
ACCCCAAGCCCAGGCTGCCAGCGGCAGAAGTATTCCATGAACAAGG
AAGGTCCTCTCTAGCCGGGCCCACACGCCCAGATTCCATAGCTCCTC
TGCAGGCCTAGGCTGCCTCTCAAGTAGGTCAAGTGGTTAAGGGCCTA
TAGGGGTCCCCACTCCCCTCTTCTCCAGGTAGGAGCCAGGTTAGATC
TGGGGCATCTGTGTGCCTGCAGCTTGCCCCAAACCACATACCTCATG
GTTTCCATCAATGGCTGGGTGCCACTTAACCCTGACCTGGCCCCAGA
CCTCCTCCTTCATCTGGTGACAGTATCCGGGAGTCCAGCAAAAGCCC
TGCTGCCCAGGTCTGCTGTATCCATATTCCAAGGGGATAGACCCTGC
AGGTCCCTACCCCTGTAAATCACCTGCCAGCCTCGCAGCC.

9. The isolated nucleic acid molecule of claim 1, wherein the nucleic acid sequence is 95% identical to the following sequence when optimally aligned:
(SEQ ID NO: 3)
GGCAGCCACATGCCTTTCATAGATGCAGAATTTCTGTCATAGCAAGT
CCACAGGCTACACCATGGGCCTTTTTGTTTCCTGTGCCCTCCCAGGTG
ACCCCAAGCCCAGGCTGCCAGCGGCAGAAGTATTCCATGAACAAGG
AAGGTCCTCTCTAGCCGGGCCCACACGCCCAGATTCCATAGCTCCTC
TGCAGGCCTAGGCTGCCTCTCAAGTAGGTCAAGTGGTTAAGGGCCTA
TAGGGGTCCCCACTCCCCTCTTCTCCAGGTAGGAGCCAGGTTAGATC
TGGGGCATCTGTGTGCCTGCAGCTTGCCCCAAACCACATACCTCATG
GTTTCCATCAATGGCTGGGTGCCACTTAACCCTGACCTGGCCCCAGA
CCTCCTCCTTCATCTGGTGACAGTATCCGGGAGTCCAGCAAAAGCCC
TGCTGCCCAGGTCTGCTGTATCCATATTCCAAGGGGATAGACCCTGC
AGGTCCCTACCCCTGTAAATCACCTGCCAGCCTCGCAGCC.

10. The isolated nucleic acid molecule of claim 1, wherein the nucleic acid sequence is 90% identical to the following sequence when optimally aligned:
(SEQ ID NO: 3)
GGCAGCCACATGCCTTTCATAGATGCAGAATTTCTGTCATAGCAAGT
CCACAGGCTACACCATGGGCCTTTTTGTTTCCTGTGCCCTCCCAGGTG
ACCCCAAGCCCAGGCTGCCAGCGGCAGAAGTATTCCATGAACAAGG
AAGGTCCTCTCTAGCCGGGCCCACACGCCCAGATTCCATAGCTCCTC
TGCAGGCCTAGGCTGCCTCTCAAGTAGGTCAAGTGGTTAAGGGCCTA
TAGGGGTCCCCACTCCCCTCTTCTCCAGGTAGGAGCCAGGTTAGATC
TGGGGCATCTGTGTGCCTGCAGCTTGCCCCAAACCACATACCTCATG
GTTTCCATCAATGGCTGGGTGCCACTTAACCCTGACCTGGCCCCAGA
CCTCCTCCTTCATCTGGTGACAGTATCCGGGAGTCCAGCAAAAGCCC
TGCTGCCCAGGTCTGCTGTATCCATATTCCAAGGGGATAGACCCTGC
AGGTCCCTACCCCTGTAAATCACCTGCCAGCCTCGCAGCC.

11. The isolated nucleic acid molecule of claim 1, wherein the nucleic acid sequence is 80% identical to the following sequence when optimally aligned:
(SEQ ID NO: 3)
GGCAGCCACATGCCTTTCATAGATGCAGAATTTCTGTCATAGCAAGT
CCACAGGCTACACCATGGGCCTTTTTGTTTCCTGTGCCCTCCCAGGTG
ACCCCAAGCCCAGGCTGCCAGCGGCAGAAGTATTCCATGAACAAGG
AAGGTCCTCTCTAGCCGGGCCCACACGCCCAGATTCCATAGCTCCTC
TGCAGGCCTAGGCTGCCTCTCAAGTAGGTCAAGTGGTTAAGGGCCTA
TAGGGGTCCCCACTCCCCTCTTCTCCAGGTAGGAGCCAGGTTAGATC
TGGGGCATCTGTGTGCCTGCAGCTTGCCCCAAACCACATACCTCATG
GTTTCCATCAATGGCTGGGTGCCACTTAACCCTGACCTGGCCCCAGA
CCTCCTCCTTCATCTGGTGACAGTATCCGGGAGTCCAGCAAAAGCCC
TGCTGCCCAGGTCTGCTGTATCCATATTCCAAGGGGATAGACCCTGC
AGGTCCCTACCCCTGTAAATCACCTGCCAGCCTCGCAGCC.

12. The isolated nucleic acid molecule of claim 1, wherein the nucleic acid sequence of mouse mouse SCE1 comprises:
(SEQ ID NO: 4)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAGGCTGCCTCTCAAGTAGGTCAAGT
GGTTAAGGGCCTATAGGGGTCCCCACTCCCCTCTTCTCCAGGTAGGA
GCCAGGTTAGATCTGGGGCATCTGTGTGCCTGCAGCTTGCCCCAAAC
CACATACCTCATGGTTTCCATCAATGGCTGGGTGCCACTTAACCCTGA
CCTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAG.

13. The isolated nucleic acid molecule of claim 1, wherein the nucleic acid sequence hybridizes under stringent conditions to the following sequence or its complement:
(SEQ ID NO: 4)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAGGCTGCCTCTCAAGTAGGTCAAGT
GGTTAAGGGCCTATAGGGGTCCCCACTCCCCTCTTCTCCAGGTAGGA
GCCAGGTTAGATCTGGGGCATCTGTGTGCCTGCAGCTTGCCCCAAAC
CACATACCTCATGGTTTCCATCAATGGCTGGGTGCCACTTAACCCTGA
CCTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAG.

14. The isolated nucleic acid molecule of claim 1, wherein the nucleic acid sequence is 95% identical to the following sequence when optimally aligned:
(SEQ ID NO: 4)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAGGCTGCCTCTCAAGTAGGTCAAGT
GGTTAAGGGCCTATAGGGGTCCCCACTCCCCTCTTCTCCAGGTAGGA
GCCAGGTTAGATCTGGGGCATCTGTGTGCCTGCAGCTTGCCCCAAAC
CACATACCTCATGGTTTCCATCAATGGCTGGGTGCCACTTAACCCTGA
CCTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAG.

15. The isolated nucleic acid molecule of claim 1, wherein the nucleic acid sequence is 90% identical to the following sequence when optimally aligned:
(SEQ ID NO: 4)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAGGCTGCCTCTCAAGTAGGTCAAGT
GGTTAAGGGCCTATAGGGGTCCCCACTCCCCTCTTCTCCAGGTAGGA
GCCAGGTTAGATCTGGGGCATCTGTGTGCCTGCAGCTTGCCCCAAAC
CACATACCTCATGGTTTCCATCAATGGCTGGGTGCCACTTAACCCTGA
CCTGGCCCCAGACCTCCTCCTTCATCTGGTGACAGTATCCGGGAGTCC
AGCAAAAGCCCTGCTGCCCAGGTCTGCTGTATCCATATTCCAAGGGG
ATAGACCCTGCAG.

16. The isolated nucleic acid molecule of claim 1, wherein the nucleic acid sequence is 80% identical to the following sequence when optimally aligned:

| | |
|---|---|
| CGGGCAGCCTGGCCGGCAGCCACATGCCTTTCATAGATGCAGAATTTCTGTC | (SEQ ID NO: 4) |
| ATAGCAAGTCCACAGGCTACACCATGGGCCTTTTTGTTTCCTGTGCCCTCCC | |
| AGGTGACCCCAAGCCCAGGCTGCCAGCGGCAGAAGTATTCCATGAACAAGG | |
| AAGGTCCTCTCTAGCCGGGCCCACACGCCCAGATTCCATAGCTCCTCTGCAG | |
| GCCTAGGCTGCCTCTCAAGTAGGTCAAGTGGTTAAGGGCCTATAGGGGTCC | |
| CCACTCCCCTCTTCTCCAGGTAGGAGCCAGGTTAGATCTGGGGCATCTGTGT | |
| GCCTGCAGCTTGCCCCAAACCACATACCTCATGGTTTCCATCAATGGCTGGG | |
| TGCCACTTAACCCTGACCTGGCCCCAGACCTCCTCCTTCATCTGGTGACAGT | |
| ATCCGGGAGTCCAGCAAAAGCCCTGCTGCCCAGGTCTGCTGTATCCATATTC | |
| CAAGGGGATAGACCCTGCAG. | |

17. The isolated nucleic acid molecule of claim 1, wherein the nucleic acid sequence of mouse mouse SCE1 comprises:
(SEQ ID NO: 5)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAG.

18. The isolated nucleic acid molecule of claim 1, wherein the nucleic acid sequence hybridizes under stringent conditions to the following sequence or its complement:
(SEQ ID NO: 5)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAG.

19. The isolated nucleic acid molecule of claim 1, wherein the nucleic acid sequence is 95% identical to the following sequence when optimally aligned:
(SEQ ID NO: 5)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAG.

20. The isolated nucleic acid molecule of claim 1, wherein the nucleic acid sequence is 90% identical to the following sequence when optimally aligned:
(SEQ ID NO: 5)
CGGGCAGCCTGGCCGGCAGCCACATGCCTTTCATAGATGCAGAATTT
CTGTCATAGCAAGTCCACAGGCTACACCATGGGCCTTTTTGTTTCCTG
TGCCCTCCCAGGTGACCCCAAGCCCAGGCTGCCAGCGGCAGAAGTAT
TCCATGAACAAGGAAGGTCCTCTCTAGCCGGGCCCACACGCCCAGAT
TCCATAGCTCCTCTGCAGGCCTAG.

21. The isolated nucleic acid molecule of claim 1, wherein the nucleic acid sequence is 80% identical to the following sequence when optimally aligned:

| | |
|---|---|
| CGGGCAGCCTGGCCGGCAGCCACATGCCTTTCATAGATGCAGAATTTCTGTC | (SEQ ID NO: 5) |
| ATAGCAAGTCCACAGGCTACACCATGGGCCTTTTTGTTTCCTGTGCCCTCCC | |
| AGGTGACCCCAAGCCCAGGCTGCCAGCGGCAGAAGTATTCCATGAACAAGG | |
| AAGGTCCTCTCTAGCCGGGCCCACACGCCCAGATTCCATAGCTCCTCTGCAG | |
| GCCTAG. | |

22. The isolated nucleic acid molecule of claim 1, wherein the nucleic acid sequence comprises:

| | |
|---|---|
| CGGGAAGCCTGGCAGGCAGCCACATGCCTCTCATAGATGCAGAATTTCTGC | (SEQ ID NO: 6) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAAGCCTCTCCCCATCCATGAGAGAGGTCACCTG | |
| CGGCCCACTCTGCAGCCAGTGTACAGCCTGTCCAAGGTGGCCAAATGGAGA | |
| GGCACGGACCAGGAGCCCATTAGAGCCAGCAGAGCTGCACGGGCAATGCC | |
| CTCC. | |

23. The isolated nucleic acid molecule of claim, wherein the nucleic acid sequence hybridizes under stringent conditions to the following sequence herein or its complement:

| | |
|---|---|
| CGGGAAGCCTGGCAGGCAGCCACATGCCTCTCATAGATGCAGAATTTCTGC | (SEQ ID NO: 6) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAAGCCTCTCCCCATCCATGAGAGAGGTCACCTG | |
| CGGCCCACTCTGCAGCCAGTGTACAGCCTGTCCAAGGTGGCCAAATGGAGA | |
| GGCACGGACCAGGAGCCCATTAGAGCCAGCAGAGCTGCACGGGCAATGCC | |
| CTCC. | |

24. The isolated nucleic acid molecule of claim 1, wherein the nucleic acid sequence is 95% identical to the following sequence when optimally aligned:

| | |
|---|---|
| CGGGAAGCCTGGCAGGCAGCCACATGCCTCTCATAGATGCAGAATTTCTGC | (SEQ ID NO: 6) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAAGCCTCTCCCCATCCATGAGAGAGGTCACCTG | |
| CGGCCCACTCTGCAGCCAGTGTACAGCCTGTCCAAGGTGGCCAAATGGAGA | |
| GGCACGGACCAGGAGCCCATTAGAGCCAGCAGAGCTGCACGGGCAATGCC | |
| CTCC. | |

25. The nucleic acid molecule of claim 1, wherein the nucleic acid sequence is 90% identical to the following sequence when optimally aligned:

| | |
|---|---|
| CGGGAAGCCTGGCAGGCAGCCACATGCCTCTCATAGATGCAGAATTTCTGC | (SEQ ID NO: 6) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAAGCCTCTCCCCATCCATGAGAGAGGTCACCTG | |
| CGGCCCACTCTGCAGCCAGTGTACAGCCTGTCCAAGGTGGCCAAATGGAGA | |
| GGCACGGACCAGGAGCCCATTAGAGCCAGCAGAGCTGCACGGGCAATGCC | |
| CTCC. | |

26. The nucleic acid molecule of claim 1, wherein the nucleic acid sequence is 80% identical to the following sequence when optimally aligned:

| | |
|---|---|
| CGGGAAGCCTGGCAGGCAGCCACATGCCTCTCATAGATGCAGAATTTCTGC | (SEQ ID NO: 6) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAAGCCTCTCCCCATCCATGAGAGAGGTCACCTG | |
| CGGCCCACTCTGCAGCCAGTGTACAGCCTGTCCAAGGTGGCCAAATGGAGA | |
| GGCACGGACCAGGAGCCCATTAGAGCCAGCAGAGCTGCACGGGCAATGCC | |
| CTCC. | |

27. The isolated nucleic acid molecule of claim 1, wherein the nucleic acid sequence comprises:

| | |
|---|---|
| CGGGAAGCCTGGCAGGCAGCCACATGCCTCTCATAGATGCAGAATTTCTGC | (SEQ ID NO: 7) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAA. | |

28. The isolated nucleic acid molecule of claim 1, wherein the nucleic acid sequence hybridizes under stringent conditions to the following sequence herein or its complement:

| | |
|---|---|
| CGGGAAGCCTGGCAGGCAGCCACATGCCTCTCATAGATGCAGAATTTCTGC | (SEQ ID NO: 7) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAA. | |

29. The isolated nucleic acid molecule of claim 1, wherein the nucleic acid sequence is 95% identical to the following sequence when optimally aligned:

| | |
|---|---|
| CGGGAAGCCTGGCAGGCAGCCACATGCCTCTCATAGATGCAGAATTTCTGC | (SEQ ID NO: 7) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAA. | |

30. The nucleic acid molecule of claim 1, wherein the nucleic acid sequence is 90% identical to the following sequence when optimally aligned:

| | |
|---|---|
| CGGGAAGCCTGGCAGGCAGCCACATGCCTCTCATAGATGCAGAATTTCTGC | (SEQ ID NO: 7) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAA. | |

31. The nucleic acid molecule of claim 1, wherein the nucleic acid sequence is 80% identical to the following sequence when optimally aligned:

| | |
|---|---|
| CGGGAAGCCTGGCAGGCAGCCACATGCCTCTCATAGATGCAGAATTTCTGC | (SEQ ID NO: 7) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCGGCCTCCTTGAGAGGGGCCAGGAGAGTGGAGGCCCTGCGTGCCCCCGG | |
| CGCCTTCTCCAGTGGGACAAGGGGCCACAGCTTAAAGCTCGGTTCTGCACCT | |
| TTAGCCCCAAGCTCTCCTCACCTCCACCCCCAGAAAACACACTTGTTGTCGT | |
| TGCCATCGCTCAAAGTTTCTGTCCGACTCTTCACAGGCAGGGAGCAGACTAT | |
| CTATCTGCTGGACAGGAGCTGGAGCTGAGTGCTCGACAAAAGCCCTGGCTC | |
| CCACTGGGCACAGCCTGCAA. | |

32. The isolated nucleic acid molecule of claim 1, wherein the nucleic acid sequence comprises:

| | |
|---|---|
| CGGGAAGCCTGGCAGGCAGCCACATGCCTCTCATAGATGCAGAATTTCTGC | (SEQ ID NO: 8) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCG. | |

33. The isolated nucleic acid molecule of claim 1, wherein the nucleic acid sequence hybridizes under stringent conditions to the following sequence herein or its complement:

| | |
|---|---|
| CGGGAAGCCTGGCAGGCAGCCACATGCCTCTCATAGATGCAGAATTTCTGC | (SEQ ID NO: 8) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCG. | |

34. The isolated nucleic acid molecule of claim 1, wherein the nucleic acid sequence is 95% identical to the following sequence when optimally aligned:

| | |
|---|---|
| CGGGAAGCCTGGCAGGCAGCCACATGCCTCTCATAGATGCAGAATTTCTGC | (SEQ ID NO: 8) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCG. | |

35. The nucleic acid molecule of claim 1, wherein the nucleic acid sequence is 90% identical to the following sequence when optimally aligned:

| | |
|---|---|
| CGGGAAGCCTGGCAGGCAGCCACATGCCTCTCATAGATGCAGAATTTCTGC | (SEQ ID NO: 8) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCG. | |

36. The nucleic acid molecule of claim 1, wherein the nucleic acid sequence is 80% identical to the following sequence when optimally aligned:

| | |
|---|---|
| CGGGAAGCCTGGCAGGCAGCCACATGCCTCTCATAGATGCAGAATTTCTGC | (SEQ ID NO: 8) |
| CATCGCAAATCCACAGGCCACACCATGGCCCTTTTTGTTTCCTTTGCCCTCCC | |
| TGGTGACCCCAAGCACAGCGCGCCGTGACACAAGTATTCCACAAACAAGGA | |
| GCGTCCTCTTTGGCCCTGGCCCACACTTCCAAATTCCATGGTCTCTGCAGGG | |
| CTCG. | |

37. An isolated nucleic acid molecule comprising a nucleic acid sequence of SCE1 consisting of a consensus sequence of mouse SCE1 and human SCE1, wherein the consensus SCE1 enhances transcription of operably-linked sequences in Schwann cells.

38. The isolated nucleic acid molecule of claim 37, wherein the nucleic acid sequence of consensus SCE1 comprises:

| | |
|---|---|
| 5′-CGGSAAG | (SEQ ID NO: 9) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCARGYCYCTMCCCMT | |
| SYATGAGAGARRTCACCTGCSRSCCWCKCWGCMGSCASYSTMSMRCCYRYCCAMG | |
| STKRMYRRATRKRGAGRCAYKGACCASGMGYCYWYTWGRSYMRRSAGMKCTGMR | |
| CRGSCWMTSCCSWSC - 3′ | |
| or - | |
| 5′- CGGSAG | (SEQ ID NO: 10) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCARGYCYCTMCCCMTS | |
| YARRTCACCTGCSRSCCWCKCWGCMGSCASYSTMSMRCCYRYCCAMGSTKRMYRR | |
| ATRKRGAGRCAYKGAGCCASGMGYCYWYTWGRSYMRRSAGMKCTGMRCRGSCW | |
| MTSCCSWSC - 3′. | |

39. The isolated nucleic acid molecule of claim 37, wherein the nucleic acid sequence of consensus SCE1 hybridizes under stringent conditions to the following sequence or its complement:

| | |
|---|---|
| 5′-CGGSAAG | (SEQ ID NO: 9) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCARGYCYCTMCCCMT | |
| SYATGAGAGARRTCACCTGCSRSCCWCKCWGCMGSCASYSTMSMRCCYRYCCAMG | |
| STKRMYRRATRKRGAGRCAYKGACCASGMGYCYWYTWGRSYMRRSAGMKCTGMR | |
| CRGSCWMTSCCSWSC - 3′ | |
| or - | |
| 5′- CGGSAG | (SEQ ID NO: 10) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCARGYCYCTMCCCMTS | |
| YARRTCACCTGCSRSCCWCKCWGCMGSCASYSTMSMRCCYRYCCAMGSTKRMYRR | |
| ATRKRGAGRCAYKGAGCCASGMGYCYWYTWGRSYMRRSAGMKCTGMRCRGSCW | |
| MTSCCSWSC - 3′. | |

40. The isolated nucleic acid molecule of claim 37, wherein the nucleic acid sequence of consensus SCE1 is 95% identical to the following sequence when optimally aligned:

| | |
|---|---|
| 5′-CGGSAAG | (SEQ ID NO: 9) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCARGYCYCTMCCCMT | |
| SYATGAGAGARRTCACCTGCSRSCCWCKCWGCMGSCASYSTMSMRCCYRYCCAMG | |
| STKRMYRRATRKRGAGRCAYKGACCASGMGYCYWYTWGRSYMRRSAGMKCTGMR | |
| CRGSCWMTSCCSWSC - 3′ | |
| or - | |
| 5′- CGGSAG | (SEQ ID NO: 10) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCARGYCYCTMCCCMTS | |
| YARRTCACCTGCSRSCCWCKCWGCMGSCASYSTMSMRCCYRYCCAMGSTKRMYRR | |
| ATRKRGAGRCAYKGAGCCASGMGYCYWYTWGRSYMRRSAGMKCTGMRCRGSCW | |
| MTSCCSWSC - 3′. | |

41. The isolated nucleic acid molecule of claim 37, wherein the nucleic acid sequence of consensus SCE 1 is 90% identical to the following sequence when optimally aligned:

| | |
|---|---|
| 5′- | (SEQ ID NO: 9) |
| CGGSAAGCCTGGCMGGCAGCCACATGCCTYTCATAGATGCAGAATTTCTGYCATM | |
| GCAARTCCACAGGCYACACCATGGSCCTTTTTGTTTCCTKTGCCCTCCCWGGTGAC | |
| CCCAAGCMCAGSSYGCCGYGRCASAAGTATTCCAYRAACAAGGARSGTCCTCTYTR | |
| GCCSTGGCCCACACKYCCARATTCCATRGYCTCTGCAGGSCTMGGCYKCCTYKMRA | |
| GRGGYCARGWGRKTRRRGGCCYWKMGKGSYCCCSRCKCCTTCTCCAGKKRGRMR | |
| MSRGGYYASAKCTKRRRSMTCKGTTSYSYRCMKYTWGCCCCAARCYMYMYWCMY | |
| CWYSRYYYCCAKMAAWSRCACTKGKTGYCRYTKMMMYCKSWCMWRGYYYCWG | |
| WCCGAYYCTYCWYMRKCWGGKRRCAGACTATCYRKSWGYYSRRCARRAGCYSKR | |
| SYKSMSWGSTCKRCWRWAKCCMTRGYTCCMASKGGRYASASCCTGCARGYCYCT | |
| MCCCMTSYATGAGAGARRTCACCTGCSRSCCWCKCWGCMGSCASYSTMSMRCCYR | |
| YCCAMGSTKRMYRRATRKRGAGRCAYKGACCASGMGYCYWYTWGRSYMRRSAG | |
| MKCTGMRCRGSCWMTSCCSWSC - 3′ | |
| or - | |
| 5′- | (SEQ ID NO: 10) |
| CGGSAGCCTGGCMGGCAGCCACATGCCTYTCATAGATGCAGAATTTCTGYCATMG | |
| CAARTCCACAGGCYACACCATGGSCCTTTTTGTTTCCTKTGCCCTCCCWGGTGACC | |
| CCAAGCMCAGSSYGCCAGYGRCASAAGTATTCCAYRAACAAGGARSGTCCTCTYTR | |
| GCCSGGCCCACACKYCCARATTCCATRGYTCCTCTGCAGGSCTMGGCYKCCTYKMR | |
| AGTRGGYCARGWGRKTRRRGGCCYWKMGKGSYCCCSRCKCCCCTCTTCTCCAGKK | |
| RGRMRMSRGGYYASAKCTKRRRSMTCKGTGTSYSYRCMKYTWGCCCCAARCYMY | |
| MYWCMYCWYSRYYYCCAKMAAWSRCTKGKTGYCRYTKMMMYCKSWCMWRGYY | |
| YCWGWCCYYCTYCWYMRKCWGGKRRCAGTATCYRKSWGYYSRRCARRAGCYSKR | |
| SYKSMSWGSTCKRCWRWAKCCMTRYTCCMASKGGRYASASCCTGCARGYCYCTMC | |
| CCMTSYARRTCACCTGCSRSCCWCKCWGCMGSCASYSTMSMRCCYRYCCAMGSTK | |
| RMYRRATRKRGAGRCAYKGAGCCASGMGYCYWYTWGRSYMRRSAGMKCTGMRC | |
| RGSCWMTSCCSWSC - 3′. | |

42. The isolated nucleic acid molecule of claim 37, wherein the nucleic acid sequence of consensus SCE1 is 80% identical to the following sequence when optimally aligned:

| | |
|---|---|
| 5′- CGGSAAG | (SEQ ID NO: 9) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCARGYCYCTMCCCMT | |
| SYATGAGAGARRTCACCTGCSRSCCWCKCWGCMGSCASYSTMSMRCCYRYCCAMG | |
| STKRMYRRATRKRGAGRCAYKGACCASGMGYCYWYTWGRSYMRRSAGMKCTGMR | |
| CRGSCWMTSCCSWSC - 3′ | |
| or - | |
| 5′- CGGSAG | (SEQ ID NO: 10) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCARGYCYCTMCCCMTS | |
| YARRTCACCTGCSRSCCWCKCWGCMGSCASYSTMSMRCCYRYCCAMGSTKRMYRR | |
| ATRKRGAGRCAYKGAGCCASGMGYCYWYTWGRSYMRRSAGMKCTGMRCRGSCW | |
| MTSCCSWSC - 3′. | |

43. The isolated nucleic acid molecule of claim 37, wherein the nucleic acid sequence of consensus SCE1 comprises:

| | |
|---|---|
| 5′- CGGSAAG | (SEQ ID NO: 11) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCAR - 3′ | |
| or - | |
| 5′- CGGSAG | (SEQ ID NO: 12) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCAR - 3′. | |

44. The isolated nucleic acid molecule of claim 37, wherein the nucleic acid sequence of consensus SCE1 hybridizes under stringent conditions to the following sequence or its complement:

| | |
|---|---|
| 5′- CGGSAAG | (SEQ ID NO: 11) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCAR - 3′ | |
| or - | |
| 5′- CGGSAG | (SEQ ID NO: 12) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCAR - 3′. | |

45. The isolated nucleic acid molecule of claim 37, wherein the nucleic acid sequence of consensus SCE1 is 95% identical to the following sequence when optimally aligned:

| | |
|---|---|
| 5′- CGGSAAG | (SEQ ID NO: 11) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCAR - 3′ | |
| or - | |
| 5′- CGGSAG | (SEQ ID NO: 12) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCAR - 3′. | |

46. The isolated nucleic acid molecule of claim 37, wherein the nucleic acid sequence of consensus SCE1 is 90% identical to the following sequence when optimally aligned:

| | |
|---|---|
| 5′- CGGSAAG | (SEQ ID NO: 11) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCAR - 3′ | |
| or - | |
| 5′- CGGSAG | (SEQ ID NO: 12) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCAR - 3′. | |

47. The isolated nucleic acid molecule of claim 37, wherein the nucleic acid sequence of consensus SCE1 is 80% identical to the following sequence when optimally aligned:

| | |
|---|---|
| 5′- CGGSAAG | (SEQ ID NO: 11) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMGGCYKCCTYKMRAGRGGYC | |
| ARGWGRKTRRRGGCCYWKMGKGSYCCCSRCKCCTTCTCCAGKKRGRMRMSRGGY | |
| YASAKCTKRRRSMTCKGTTSYSYRCMKYTWGCCCCAARCYMYMYWCMYCWYSRY | |
| YYCCAKMAAWSRCACTKGKTGYCRYTKMMMYCKSWCMWRGYYYCWGWCCGAY | |
| YCTYCWYMRKCWGGKRRCAGACTATCYRKSWGYYSRRCARRAGCYSKRSYKSMS | |
| WGSTCKRCWRWAKCCMTRGYTCCMASKGGRYASASCCTGCAR - 3′ | |
| or - | |
| 5′- CGGSAG | (SEQ ID NO: 12) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMGGCYKCCTYKMRAGTRG | |
| GYCARGWGRKTRRRGGCCYWKMGKGSYCCCSRCKCCCCTCTTCTCCAGKKRGRMR | |
| MSRGGYYASAKCTKRRRSMTCKGTGTSYSYRCMKYTWGCCCCAARCYMYMYWCM | |
| YCWYSRYYYCCAKMAAWSRCTKGKTGYCRYTKMMMYCKSWCMWRGYYYCWGW | |
| CCYYCTYCWYMRKCWGGKRRCAGTATCYRKSWGYYSRRCARRAGCYSKRSYKSMS | |
| WGSTCKRCWRWAKCCMTRYTCCMASKGGRYASASCCTGCAR - 3′. | |

48. The isolated nucleic acid molecule of claim 37, wherein the nucleic acid sequence of consensus SCE1 comprises:

| | |
|---|---|
| CGGSAAG | (SEQ ID NO: 13) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMG - 3′ | |
| or - | |
| 5′- CGGSAG | (SEQ ID NO: 14) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMG - 3′. | |

49. The isolated nucleic acid molecule of claim 37, wherein the nucleic acid sequence of consensus SCE1 hybridizes under stringent conditions to the following sequence or its complement:

| | |
|---|---|
| CGGSAAG | (SEQ ID NO: 13) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMG - 3′ | |
| or - | |
| 5′- CGGSAG | (SEQ ID NO: 14) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMG - 3′. | |

50. The isolated nucleic acid molecule of claim 37, wherein the nucleic acid sequence of consensus SCE1 is 95% identical to the following sequence when optimally aligned:

| | |
|---|---|
| CGGSAAG | (SEQ ID NO: 13) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMG - 3′ | |
| or - | |
| 5′- CGGSAG | (SEQ ID NO: 14) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMG - 3′. | |

51. The isolated nucleic acid molecule of claim 37, wherein the nucleic acid sequence of consensus SCE1 is 90% identical to the following sequence when optimally aligned:

| | |
|---|---|
| CGGSAAG | (SEQ ID NO: 13) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMG - 3′ | |
| or - | |
| 5′- CGGSAG | (SEQ ID NO: 14) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMG - 3′. | |

52. The isolated nucleic acid molecule of claim 37, wherein the nucleic acid sequence of consensus SCE1 is 80% identical to the following sequence when optimally aligned:

| | |
|---|---|
| 5′- CGGSAAG | (SEQ ID NO: 13) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCGYGRCASAAGTATTCCAYRAACAAGGARSGTCCTCTYTRGCCSTGG | |
| CCCACACKYCCARATTCCATRGYCTCTGCAGGSCTMG - 3′ | |
| or - | |
| 5′- CGGSAG | (SEQ ID NO: 14) |
| CCTGGCMGGCAGCCACATGCCTYTCATAGATGCAGAATTTCTGYCATMGCAARTC | |
| CACAGGCYACACCATGGSCCTTTTTGTTTCCTKTGCCCTCCCWGGTGACCCCAAGC | |
| MCAGSSYGCCAGYGRCASAAGTATTCCAYRAACAAGGARSGTCCTCTYTRGCCSGG | |
| CCCACACKYCCARATTCCATRGYTCCTCTGCAGGSCTMG - 3′. | |

53. An isolated nucleic acid molecule comprising contiguous nucleic acid sequences of mouse SCE1 and mouse SCE2, wherein the contiguous nucleic acid sequences of mouse SCE1 and mouse SCE2 enhance transcription of operably-linked sequences in Schwann cells

54. The isolated nucleic acid molecule of claim 53, wherein the contiguous nucleic acid sequence of mouse SCE1 and mouse SCE2 comprises:

| | |
|---|---|
| GGATCCAGCCCCAAGGGTCTACAAAGCAGGCTCAGGGTCTACTTACAGTGG | (SEQ ID NO: 15) |
| TAAAGGCAATAGCATTTCCTTCTTCTGGTGGCCCTGCTCCCTCATTTCCTATT | |
| GTATCAGAGCTGTTTCCTAATGAAGACATCCATGCTGGGTGTCTGTCACTGA | |
| CAGAAGCACTTGTTGAAATACCCTGCAGAAAACAACTTCTTATGACAGTGG | |
| TCATCTTATTTGCTCAGTATTTAGTGAGCATCTACTTACTATTTGCACACCAC | |
| TCCAGTAAGAGAGGGGAACAAACTTCTAAAGGACTCGGGGTAGTGATGTGT | |
| GTGAGCGAGCAGACAAAACACAGACCCTGAGGGTGCCCATAGGACAGGCC | |
| TCATAGGTCGGATGAGTGAAGTCAAACAAACAACACCGGAGTCTGAGGAA | |
| GAGAAGACAACCACGTGCACGGTTCCATCTCACTGAAGCTGTGGCTCCATG | |
| CCGCCTGTCCTCAGCTGATCATCACCTGGGAGAACAATGCCCAGACAGATA | |
| AACAAGGGAAATGAGTCCTTGGACTCAGAGTCCCCTGCACCTCAGAACCTC | |
| TGAGGGGCCTGGGGATGCAGAATCATCTTCCTACCACTTAGCCAGAGAGCA | |
| GGAAGGAAGATGGTTGAGGCAGGTTCAAGGCTTCCTTGCACTGCAGAGTGA | |
| GTTCAAAGCCAGCCTGAACAAATTAATGAACTACAGTAATACAATGTAGTA | |
| CAGTACAGAACTACACAGGAAAGATTAGCTTCTCACTACACGCAGTGGCCC | |
| AGCATTGCAGGAAGTGGAGCAGGGAATGCTGGGAGAGCAAGGACATGGGA | |
| GGCGTGGGGCACAGCCATGGAAGGCAGCAGATATAGAAGCAGAAGTCCCC | |
| TGAAGCCTGATGTGAGATGGAGAGGCCTGAGCAAGGAAGAGCCCATGTCCC | |
| ACGGAGAAGGACTTAACAGGAGGGAGCAGGGAAAGGTTAAGAAGGGAGA | |
| GCCAGCATGCATGCTGGCATGGCACTCAGAGGAGACCTGCTGAGGCTCCCT | |
| GTCCCCAGCATCGTTTCAGTGGGAGGCAGATGACTGACATCAGTGGTGATA | |
| AGGAGTGACAAGGAAGGAAGGGTGACAAGGCCCATCCCATGGGCTCTGTC | |
| AGTGGAAACTGGGAGACTGTTGGTCAGGAACCTCTTAGCTCCCTGGAGCTC | |
| TCAGCAGGGCCCACAAGTCGCAGGCACGCAGTCAGGACAGCACTGAGTTGC | |
| AAGTCCAGATTCATATGCTCACAAGCCACAGCCTCCCAGGAGTCTGGAGGG | |
| CTGAATGGAGTGAGTCCTGGTCACCTTCAACTTCTGGTGTTGCTGACAACAC | |
| ATGGCGTCCCTGGACTTATGGTTGCAGTTGATGTTGCCTAGGGTCCGTAGTG | |
| GTCCCTTCTTTGTGCATGAGTTTCTTTCATCTCACAAGGAAATACCCATTTGA | |
| CTGGCATCCATCCGAATCCAGTGAAAGTTCCTCTTTACCTAATAACATCTTC | |
| TCAGACCCTATTTCCAAAGTCACATTGACCGGTGGACAAGCATGGTTCTGGG | |
| GGTGCTGTCACCCTAGGGTAGCACTGTATGTCCCAATGAAGGTGGGGGAAC | |
| AGAGGTATACACTGGATCTGTTGACTTCCTTGAAGGAAGTCGAATGATGCA | |
| CAGTCATGGGGGAAGTGGGTGCCTGGAGCCTGACAAAAGAAGTCTCTTCAG | |
| TCCACCATAAAAGACAGTTGAAGAAGGATGTGGACTGGGATTGTACTTTTA | |
| TTTATGGTGACAGTGACTACCGGGATAGTGGTGGTGGGTCTTAAGTAGGAC | |
| ATGGAAGACAAAAGGACCTTTGGAGCCTAGGGATGGGGGTTGGGATTGTGC | |
| CATCTCAGACCGGATGCTATCCTGGGGCCACACAATCCAGGCCTCAGTCTTT | |
| CTGTGGGACTGAAAGCCAGAACTTTTGGTAGATTTTGGAGATGTAAAAGTA | |
| ATGACACTTGAAGAGGCAAAGAAAAAGAAGATGTAAAAGTGTGGCCAGGT | |
| GGTGGGCCAAGCAGTGTGTGTGTGTGTGTGCATGGATTAGGTGGTAGGCTA | |
| GCGAATGGATGCATGGGGAGATAGATAATGGATGGAGGATAGGTAGATGA | |
| TAGATAGATAGATAGATAGATAGATAGATAGATAGATAGATAGACAGACA | |
| GACAGTGGATGGATGGATGGATGGATGGATGGATGGGCAGATAGATGGAC | |
| AGATGGGTGGAGTGGATAAGTGGTAGATGGGGTAAATACATATCAAGGATA | |
| AGAATTTTTATGACCAAATGCTCTAATGACTTATCAAAACAAGACTATGAG | |
| GAAGCCCTGGATACTCAGGCAATTTTGCAGATGAGGTCGCCAAGCCTTAAA | |
| GACATGAAATCACTTGTTGTAGGTAGTGGAGCTGAGAACTCAGCCCTGGCT | |
| TATTCCAGGCCCCTGGCTCATATGTTCATATTCAGTTGAATTTTCTCCTGGTG | |
| TGTGCTGTGAACTGGCTCTGTAAAATGCTGACAAGATCTGGGAATCACCTTC | |
| AGCTGTGGGCTACCTGGGACGTAGCTTTTGCTGTTGGGGGGGTGGCGGGGG | |
| ACACTGCTCTGTATCTGCGGATTAGTACAGAGCTGCTGTGTCACCCATATTA | |
| GGAGAAACTGTGTGACTATGGGGCTCCGTTTTACCACTCATGTCTACACTGG | |
| CTGCAGTAGGGTAGCTGGATGGTGGCTCTGACCCTCCTTGGGCAAAGCAAC | |
| CTCTTACTCAGCAAGACTCAGGCTGGCCAAGTATGTGGGTAGGCTGGGAGA | |
| AATACACTCCAGCGCTTGCCTGAGGCTCTCCCGTTATTGTGACCCCTTTCTC | |
| GATGTGGGAGGGTCCCTGAGTGAGCTATTTAGAGTACATAAAAACTATATG | |
| TTCCGAGCACACAAAAGAGGCATTCGGTGTGTGGTGGGTGGGTTGACAAGA | |
| TTCGTTTGTGGAAGTCCTTGAAGCTATCGCCGCGGGCAGCCTGGCCGGCAGC | |
| CACATGCCTTTCATAGATGCAGAATTTCTGTCATAGCAAGTCCACAGGCTAC | |
| ACCATGGGCCTTTTTGTTTCCTGTGCCCTCCCAGGTGACCCCAAGCCCAGGC | |
| TGCCAGCGGCAGAAGTATTCCATGAACAAGGAAGGTCCTCTCTAGCCGGGC | |
| CCACACGCCCAGATTCCATAGCTCCTCTGCAGGCCTAGGCTGCCTCTCAAGT | |
| AGGTCAAGTGGTTAAGGGCCTATAGGGGTCCCCACTCCCCTCTTCTCCAGGT | |
| AGGAGCCAGGTTAGATCTGGGGCATCTGTGTGCCTGCAGCTTGCCCCAAAC | |
| CACATACCTCATGGTTTCCATCAATGGCTGGGTGCCACTTAACCCTGACCTG | |
| GCCCCAGACCTCCTCCTTCATCTGGTGACAGTATCCGGGAGTCCAGCAAAA | |
| GCCCTGCTGCCCAGGTCTGCTGTATCCATATTCCAAGGGGATAGACCCTGCA | |
| GGTCCCTACCCCTGTAAATCACCTGCCAGCCTCGCAGCCGGCACCCTCGCAC | |
| CCACCCACGCTTAATGGATATGGAGACATTGAGCCACGCGTCTTCTTGGCTA | |
| GAGAGCTC. | |

55. The isolated nucleic acid molecule of claim 53, wherein the contiguous nucleic acid sequence of mouse SCE1 and mouse SCE2 comprises:

| | |
|---|---|
| GTGGGTAGGCTGGGAGAAATACACTCCAGCGCTTGCCTGAGGCTCTCCCGT | (SEQ ID NO: 16) |
| TATTGTGACCCCTTTCTCGATGTGGGAGGGTCCCTGAGTGAGCTATTTAGAG | |
| TACATAAAAACTATATGTTCCGAGCACACAAAAGAGGCATTCGGTGTGTGG | |
| TGGGTGGGTTGACAAGATTCGTTTGTGGAAGTCCTTGAAGCTATCGCCGCGG | |
| GCAGCCTGGCCGGCAGCCACATGCCTTTCATAGATGCAGAATTTCTGTCATA | |
| GCAAGTCCACAGGCTACACCATGGGCCTTTTTGTTTCCTGTGCCCTCCCAGG | |
| TGACCCCAAGCCCAGGCTGCCAGCGGCAGAAGTATTCCATGAACAAGGAAG | |
| GTCCTCTCTAGCCGGGCCCACACGCCCAGATTCCATAGCTCCTCTGCAGGCC | |
| TAG. | |

56. A recombinant nucleic acid molecule comprising an enhancer sequence having the sequence of the nucleic acid molecule of claim 1 operably linked to a heterologous promoter sequence.

57. The recombinant nucleic acid molecule of claim 56, further comprising a heterologous coding sequence operably-linked to the heterologous promoter sequence and the enhancer sequence.

58. An expression vector comprising the recombinant nucleic acid molecule of claim 57.

59. A method for regulating gene expression in Schwann cells, comprising:
transforming a host cell with a vector of claim 58; and
subjecting a host cell to conditions which allow expression of the heterologous coding sequence.

60. A method for treating a disease in a subject, comprising administering to the subject the vector of claim 58.

61. A transgenic cell comprising the recombinant nucleic acid molecule of claim 57.

62. The transgenic cell of claim 61, wherein the cell is a mammalian cell.

63. A non-human transgenic animal comprising the recombinant nucleic acid molecule of claim 57.

64. The non-human transgenic animal of claim 63, wherein the animal is a mouse.

65. A method for producing a non-human transgenic animal comprising introducing into the animal the isolated nucleic acid molecule of claim 57.

66. A method of isolating a nucleic acid molecule having Schwann-cell specific enhancer activity, comprising hybridizing under stringent conditions a nucleic acid preparation with a probe comprising the isolated nucleic acid molecule of claim 2.

67. A method of isolating a nucleic acid molecule having Schwann-cell specific enhancer activity, comprising hybridizing under stringent conditions a nucleic acid preparation with a probe comprising the isolated nucleic acid molecule of claim 27.

68. A method of isolating a nucleic acid molecule having Schwann-cell specific enhancer activity, comprising hybridizing under stringent conditions a nucleic acid preparation with a probe comprising the isolated nucleic acid molecule of claim 38.

69. A method of isolating a nucleic acid molecule having Schwann-cell specific enhancer activity, comprising hybridizing under stringent conditions a nucleic acid preparation with a probe comprising the isolated nucleic acid molecule of claim 7.

70. A method of isolating a nucleic acid molecule having Schwann-cell specific enhancer activity, comprising hybridizing under stringent conditions a nucleic acid preparation with a probe comprising the isolated nucleic acid molecule of claim 12.

71. A method of isolating a nucleic acid molecule having Schwann-cell specific enhancer activity, comprising hybridizing under stringent conditions a nucleic acid preparation with a probe comprising the isolated nucleic acid molecule of claim 17.

72. A method of isolating a nucleic acid molecule having Schwann-cell specific enhancer activity, comprising hybridizing under stringent conditions a nucleic acid preparation with a probe comprising the isolated nucleic acid molecule of claim 43.

73. A method of isolating a nucleic acid molecule having Schwann-cell specific enhancer activity, comprising hybridizing under stringent conditions a nucleic acid preparation with a probe comprising the isolated nucleic acid molecule of claim 48.


SEQUENCE LISTING (23 sequences)

SEQ ID NO: 1 - DNA, 267 bp, Mus musculus
gtgaagactccttaaaggcgcagggcggcgagcaggtcaccagacgctgacagctactcagaaccaaatctggttccatccagagacaagcgaagacaagagaagcagaccgagcggcgcgttcccgatcctcggccaggaccagccttccccagagcatccctgccgggacgaaccttcccaggagcatccctgccgggagcggaactttccccggagcatccagccgcggacgcagccttccagaagcacagcgcggcgccatgg

SEQ ID NO: 2 - DNA, 615 bp, Mus musculus
cggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctaggctgcctctcaagtaggtcaagtggttaagggcctataggggtccccactcccctcttctccaggtaggagccaggttagatctggggcatctgtgtgcctgcagcttgccccaaaccacatacctcatggtttccatcaatggctgggtgccacttaaccctgacctggccccagacctcctccttcatctggtgacagtatccgggagtccagcaaaagccctgctgcccaggtctgctgtatccatattccaaggggatagaccctgcaggtccctacccctgtaaatcacctgccagcctcgcagccggcaccctcgcacccacccacgcttaatggatatggagacattgagccacgcgtcttcttggctagagagctctgagcagcctctcccgagc

SEQ ID NO: 3 - DNA, 510 bp, Mus musculus
ggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctaggctgcctctcaagtaggtcaagtggttaagggcctataggggtccccactcccctcttctccaggtaggagccaggttagatctggggcatctgtgtgcctgcagcttgccccaaaccacatacctcatggtttccatcaatggctgggtgccacttaaccctgacctggccccagacctcctccttcatctggtgacagtatccgggagtccagcaaaagccctgctgcccaggtctgctgtatccatattccaaggggatagaccctgcaggtccctacccctgtaaatcacctgccagcctcgcagcc

SEQ ID NO: 4 - DNA, 486 bp, Mus musculus
cgggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctaggctgcctctcaagtaggtcaagtggttaagggcctataggggtccccactcccctcttctccaggtaggagccaggttagatctggggcatctgtgtgcctgcagcttgccccaaaccacatacctcatggtttccatcaatggctgggtgccacttaaccctgacctggccccagacctcctccttcatctggtgacagtatccgggagtccagcaaaagccctgctgcccaggtctgctgtatccatattccaaggggatagaccctgcag

SEQ ID NO: 5 - DNA, 213 bp, Mus musculus
cgggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctag

SEQ ID NO: 6 - DNA, 621 bp, Homo sapiens
cgggaagcctggcaggcagccacatgcctctcatagatgcagaatttctgccatcgcaaatccacaggccacaccatggccctttttgtttcctttgccctccctggtgaccccaagcacagcgcgccgtgacacaagtattccacaaacaaggagcgtcctctttggccctggcccacacttccaaattccatggtctctgcagggctcggcctccttgagaggggccaggagagtggaggccctgcgtgcccccggcgccttctccagtgggacaaggggccacagcttaaagctcggttctgcacctttagccccaagctctcctcacctccacccccagaaaacacacttgttgtcgttgccatcgctcaaagtttctgtccgactcttcacaggcagggagcagactatctatctgctggacaggagctggagctgagtgctcgacaaaagccctggctcccactgggcacagcctgcaagcctctccccatccatgagagaggtcacctgcggcccactctgcagccagtgtacagcctgtccaaggtggccaaatggagaggcacggaccaggagcccattagagccagcagagctgcacgggcaatgccctcc

SEQ ID NO: 7 - DNA, 485 bp, Homo sapiens
cgggaagcctggcaggcagccacatgcctctcatagatgcagaatttctgccatcgcaaatccacaggccacaccatggccctttttgtttcctttgccctccctggtgaccccaagcacagcgcgccgtgacacaagtattccacaaacaaggagcgtcctctttggccctggcccacacttccaaattccatggtctctgcagggctcggcctccttgagaggggccaggagagtggaggccctgcgtgcccccggcgccttctccagtgggacaaggggccacagcttaaagctcggttctgcacctttagccccaagctctcctcacctccacccccagaaaacacacttgttgtcgttgccatcgctcaaagtttctgtccgactcttcacaggcagggagcagactatctatctgctggacaggagctggagctgagtgctcgacaaaagccctggctcccactgggcacagcctgcaa

SEQ ID NO: 8 - DNA, 211 bp, Homo sapiens
cgggaagcctggcaggcagccacatgcctctcatagatgcagaatttctgccatcgcaaatccacaggccacaccatggccctttttgtttcctttgccctccctggtgaccccaagcacagcgcgccgtgacacaagtattccacaaacaaggagcgtcctctttggccctggcccacacttccaaattccatggtctctgcagggctcg

SEQ ID NO: 9 - DNA, 621 bp, Artificial sequence
  Artificial Sequence = human/mouse consensus SCE1 sequence
cggsaagcctggcmggcagccacatgcctytcatagatgcagaatttctgycatmgcaartccacaggcyacaccatggscctttttgtttcctktgccctcccwggtgaccccaagcmcagssygccgygrcasaagtattccayraacaaggarsgtcctctytrgccstggcccacackyccarattccatrgyctctgcaggsctmggcykcctykmragrggycargwgrktrrrggccywkmgkgsycccsrckccttctccagkkrgrmrmsrggyyasakctkrrrsmtckgttsysyrcmkytwgccccaarcymymywcmycwysryyyccakmaawsrcactkgktgycrytkmmmyckswcmwrgyyycwgwccgayyctycwymrkcwggkrrcagactatcyrkswgyysrrcarragcyskrsyksmswgstckrcwrwakccmtrgytccmaskggryasascctgcargycyctmcccmtsyatgagagarrtcacctgcsrsccwckcwgcmgscasystmsmrccyryccamgstkrmyrratrkrgagrcaykgaccasgmgycywytwgrsymrrsagmkctgmrcrgscwmtsccswsc

SEQ ID NO: 10 - DNA, 615 bp, Artificial sequence
  Artificial Sequence = mouse/human consensus SCE1 sequence
cggsagcctggcmggcagccacatgcctytcatagatgcagaatttctgycatmgcaartccacaggcyacaccatggscctttttgtttcctktgccctcccwggtgaccccaagcmcagssygccagygrcasaagtattccayraacaaggarsgtcctctytrgccsggcccacackyccarattccatrgytcctctgcaggsctmggcykcctykmragtrggycargwgrktrrrggccywkmgkgsycccsrckcccctcttctccagkkrgrmrmsrggyyasakctkrrrsmtckgtgtsysyrcmkytwgccccaarcymymywcmycwysryyyccakmaawsrctkgktgycrytkmmmyckswcmwrgyyycwgwccyyctycwymrkcwggkrrcagtatcyrkswgyysrrcarragcyskrsyksmswgstckrcwrwakccmtrytccmaskggryasascctgcargycyctmcccmtsyarrtcacctgcsrsccwckcwgcmgscasystmsmrccyryccamgstkrmyrratrkrgagrcaykgagccasgmgycywytwgrsymrrsagmkctgmrcrgscwmtsccswsc

SEQ ID NO: 11 - DNA, 485 bp, Artificial sequence
  Artificial Sequence = alternative consensus sub-sequence from human/mouse SCE1
cggsaagcctggcmggcagccacatgcctytcatagatgcagaatttctgycatmgcaartccacaggcyacaccatggscctttttgtttcctktgccctcccwggtgaccccaagcmcagssygccgygrcasaagtattccayraacaaggarsgtcctctytrgccstggcccacackyccarattccatrgyctctgcaggsctmggcykcctykmragrggycargwgrktrrrggccywkmgkgsycccsrckccttctccagkkrgrmrmsrggyyasakctkrrrsmtckgttsysyrcmkytwgccccaarcymymywcmycwysryyyccakmaawsrcactkgktgycrytkmmmyckswcmwrgyyycwgwccgayyctycwymrkcwggkrrcagactatcyrkswgyysrrcarragcyskrsyksmswgstckrcwrwakccmtrgytccmaskggryasascctgcar

SEQ ID NO: 12 - DNA, 485 bp, Artificial sequence
  Artificial Sequence = alternative consensus sub-sequence from mouse/human SCE1
cggsagcctggcmggcagccacatgcctytcatagatgcagaatttctgycatmgcaartccacaggcyacaccatggscctttttgtttcctktgccctcccwggtgaccccaagcmcagssygccagygrcasaagtattccayraacaaggarsgtcctctytrgccsggcccacackyccarattccatrgytcctctgcaggsctmggcykcctykmragtrggycargwgrktrrrggccywkmgkgsycccsrckcccctcttctccagkkrgrmrmsrggyyasakctkrrrsmtckgtgtsysyrcmkytwgccccaarcymymywcmycwysryyyccakmaawsrctkgktgycrytkmmmyckswcmwrgyyycwgwccyyctycwymrkcwggkrrcagtatcyrkswgyysrrcarragcyskrsyksmswgstckrcwrwakccmtrytccmaskggryasascctgcar

SEQ ID NO: 13 - DNA, 211 bp, Artificial sequence
  Artificial Sequence = alternative consensus sub-sequence from human/mouse SCE1
cggsaagcctggcmggcagccacatgcctytcatagatgcagaatttctgycatmgcaartccacaggcyacaccatggscctttttgtttcctktgccctcccwggtgaccccaagcmcagssygccgygrcasaagtattccayraacaaggarsgtcctctytrgccstggcccacackyccarattccatrgyctctgcaggsctmg

SEQ ID NO: 14 - DNA, 212 bp, Artificial sequence
  Artificial Sequence = alternative consensus sub-sequence from mouse/human SCE1
cggsagcctggcmggcagccacatgcctytcatagatgcagaatttctgycatmgcaartccacaggcyacaccatggscctttttgtttcctktgccctcccwggtgaccccaagcmcagssygccagygrcasaagtattccayraacaaggarsgtcctctytrgccsggcccacackyccarattccatrgytcctctgcaggsctmg

SEQ ID NO: 15 - DNA, 3543 bp, Mus musculus
  misc_feature (1)..(2946): SCE2 sequence
ggatccagccccaagggtctacaaagcaggctcagggtctacttacagtggtaaaggcaatagcatttccttcttctggtggccctgctccctcatttcctattgtatcagagctgtttcctaatgaagacatccatgctgggtgtctgtcactgacagaagcacttgttgaaataccctgcagaaaacaacttcttatgacagtggtcatcttatttgctcagtatttagtgagcatctacttactatttgcacaccactccagtaagagaggggaacaaacttctaaaggactcggggtagtgatgtgtgtgagcgagcagacaaaacacagaccctgagggtgcccataggacaggcctcataggtcggatgagtgaagtcaaacaaacaacaccggagtctgaggaagagaagacaaccacgtgcacggttccatctcactgaagctgtggctccatgccgcctgtcctcagctgatcatcacctgggagaacaatgcccagacagataaacaagggaaatgagtccttggactcagagtcccctgcacctcagaacctctgaggggcctggggatgcagaatcatcttcctaccacttagccagagagcaggaaggaagatggttgaggcaggttcaaggcttccttgcactgcagagtgagttcaaagccagcctgaacaaattaatgaactacagtaatacaatgtagtacagtacagaactacacaggaaagattagcttctcactacacgcagtggcccagcattgcaggaagtggagcagggaatgctgggagagcaaggacatgggaggcgtggggcacagccatggaaggcagcagatatagaagcagaagtcccctgaagcctgatgtgagatggagaggcctgagcaaggaagagcccatgtcccacggagaaggacttaacaggagggagcagggaaaggttaagaagggagagccagcatgcatgctggcatggcactcagaggagacctgctgaggctccctgtccccagcatcgtttcagtgggaggcagatgactgacatcagtggtgataaggagtgacaaggaaggaagggtgacaaggcccatcccatgggctctgtcagtggaaactgggagactgttggtcaggaacctcttagctccctggagctctcagcagggcccacaagtcgcaggcacgcagtcaggacagcactgagttgcaagtccagattcatatgctcacaagccacagcctcccaggagtctggagggctgaatggagtgagtcctggtcaccttcaacttctggtgttgctgacaacacatggcgtccctggacttatggttgcagttgatgttgcctagggtccgtagtggtcccttctttgtgcatgagtttctttcatctcacaaggaaatacccatttgactggcatccatccgaatccagtgaaagttcctctttacctaataacatcttctcagaccctatttccaaagtcacattgaccggtggacaagcatggttctgggggtgctgtcaccctagggtagcactgtatgtcccaatgaaggtgggggaacagaggtatacactggatctgttgacttccttgaaggaagtcgaatgatgcacagtcatgggggaagtgggtgcctggagcctgacaaaagaagtctcttcagtccaccataaaagacagttgaagaaggatgtggactgggattgtacttttatttatggtgacagtgactaccgggatagtggtggtgggtcttaagtaggacatggaagacaaaaggacctttggagcctagggatgggggttgggattgtgccatctcagaccggatgctatcctggggccacacaatccaggcctcagtctttctgtgggactgaaagccagaacttttggtagattttggagatgtaaaagtaatgacacttgaagaggcaaagaaaaagaagatgtaaaagtgtggccaggtggtgggccaagcagtgtgtgtgtgtgtgtgcatggattaggtggtaggctagcgaatggatgcatggggagatagataatggatggaggataggtagatgatagatagatagatagatagatagatagatagatagatagatagacagacagacagtggatggatggatggatggatggatggatgggcagatagatggacagatgggtggagtggataagtggtagatggggtaaatacatatcaaggataagaatttttatgaccaaatgctctaatgacttatcaaaacaagactatgaggaagccctggatactcaggcaattttgcagatgaggtcgccaagccttaaagacatgaaatcacttgttgtaggtagtggagctgagaactcagccctggcttattccaggcccctggctcatatgttcatattcagttgaattttctcctggtgtgtgctgtgaactggctctgtaaaatgctgacaagatctgggaatcaccttcagctgtgggctacctgggacgtagcttttgctgttgggggggtggcgggggacactgctctgtatctgcggattagtacagagctgctgtgtcacccatattaggagaaactgtgtgactatggggctccgttttaccactcatgtctacactggctgcagtagggtagctggatggtggctctgaccctccttgggcaaagcaacctcttactcagcaagactcaggctggccaagtatgtgggtaggctgggagaaatacactccagcgcttgcctgaggctctcccgttattgtgacccctttctcgatgtgggagggtccctgagtgagctatttagagtacataaaaactatatgttccgagcacacaaaagaggcattcggtgtgtggtgggtgggttgacaagattcgtttgtggaagtccttgaagctatcgccgcgggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctaggctgcctctcaagtaggtcaagtggttaagggcctataggggtccccactcccctcttctccaggtaggagccaggttagatctggggcatctgtgtgcctgcagcttgccccaaaccacatacctcatggtttccatcaatggctgggtgccacttaaccctgacctggccccagacctcctccttcatctggtgacagtatccgggagtccagcaaaagccctgctgcccaggtctgctgtatccatattccaaggggatagaccctgcaggtccctacccctgtaaatcacctgccagcctcgcagccggcaccctcgcacccacccacgcttaatggatatggagacattgagccacgcgtcttcttggctagagagctc

SEQ ID NO: 16 - DNA, 416 bp, Mus musculus
  misc_feature (1)..(203): 203 bp of contiguous 5′ mouse sequence from SCE2
gtgggtaggctgggagaaatacactccagcgcttgcctgaggctctcccgttattgtgacccctttctcgatgtgggagggtccctgagtgagctatttagagtacataaaaactatatgttccgagcacacaaaagaggcattcggtgtgtggtgggtgggttgacaagattcgtttgtggaagtccttgaagctatcgccgcgggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctag

SEQ ID NO: 17 - DNA, 1022 bp, Homo sapiens
  misc_feature (402)..(1022): SCE1 sequence
gtcactgtgaaattcaccaaaggctgcacaacctaaccgaatctacatgaatattgggctctttaagaatctcttatctctacgccaaccgtgttagggaagctgggcatgtggactgtcaccgcgctgaaggaggaaggaacctgagctcagtgtgtgcagcctcacaggggtgactggccagcgcatggccgcgcctcgccgaccccgtccccaccctcctgaggctcccctagcacacggctgcctctcctgtggcctcttcctgccgtgggaggtgccaggggtgagctatgtggagcaaataaaaactatatgttctgggcacacaaaggaggcattggtgtgagatgggtgggttgatgagcctcgtttgtgggaaagttttcaagaaatctctgcgggaagcctggcaggcagccacatgcctctcatagatgcagaatttctgccatcgcaaatccacaggccacaccatggccctttttgtttcctttgccctccctggtgaccccaagcacagcgcgccgtgacacaagtattccacaaacaaggagcgtcctctttggccctggcccacacttccaaattccatggtctctgcagggctcggcctccttgagaggggccaggagagtggaggccctgcgtgcccccggcgccttctccagtgggacaaggggccacagcttaaagctcggttctgcacctttagccccaagctctcctcacctccacccccagaaaacacacttgttgtcgttgccatcgctcaaagtttctgtccgactcttcacaggcagggagcagactatctatctgctggacaggagctggagctgagtgctcgacaaaagccctggctcccactgggcacagcctgcaagcctctccccatccatgagagaggtcacctgcggcccactctgcagccagtgtacagcctgtccaaggtggccaaatggagaggcacggaccaggagcccattagagccagcagagctgcacgggcaatgccctcc

SEQ ID NO: 18 - DNA, 1374 bp, Homo sapiens
  misc_feature (734)..(1354): SCE1 sequence
gaggcaggagaatcacttgaacctgggaggtggaggctacagtgagctgagatcatgcctccgcactccagcctgggtcacagagtgagactcagcctcataaaataaaaataaaaaaattaaacaaagtcagccatattggcctccagaccttggctaaaagtccaaatgcaactcagttcaactagtgtgtttgagctgccactatgtcaaattggccaagagctaggaataatttttttaagttctatgctccccctgagaagcaattcctagactacctgtgatgccactatcttatgactgctctgggtcggccaatgttggtacaagtcactgtgaaattcaccaaaggctgcacaacctaaccgaatctacatgaatattgggctctttaagaatctcttatctctacgccaaccgtgttagggaagctgggcatgtggactgtcaccgcgctgaaggaggaaggaacctgagctcagtgtgtgcagcctcacaggggtgactggccagcgcatggccgcgcctcgccgaccccgtccccaccctcctgaggctcccctagcacacggctgcctctcctgtggcctcttcctgccgtgggaggtgccaggggtgagctatgtggagcaaataaaaactatatgttctgggcacacaaaggaggcattggtgtgagatgggtgggttgatgagcctcgtttgtgggaaagttttcaagaaatctctgcgggaagcctggcaggcagccacatgcctctcatagatgcagaatttctgccatcgcaaatccacaggccacaccatggccctttttgtttcctttgccctccctggtgaccccaagcacagcgcgccgtgacacaagtattccacaaacaaggagcgtcctctttggccctggcccacacttccaaattccatggtctctgcagggctcggcctccttgagaggggccaggagagtggaggccctgcgtgcccccggcgccttctccagtgggacaaggggccacagcttaaagctcggttctgcacctttagccccaagctctcctcacctccacccccagaaaacacacttgttgtcgttgccatcgctcaaagtttctgtccgactcttcacaggcagggagcagactatctatctgctggacaggagctggagctgagtgctcgacaaaagccctggctcccactgggcacagcctgcaagcctctccccatccatgagagaggtcacctgcggcccactctgcagccagtgtacagcctgtccaaggtggccaaatggagaggcacggaccaggagcccattagagccagcagagctgcacgggcaatgccctcctcgtgccaagaggctgatgg

SEQ ID NO: 19 - DNA, 3001 bp, Mus musculus
  misc_feature (1500)..(2115): SCE1 sequence
tccagtgaaagttccttctttacctaataacatctttcagacctatttccaaagtcacattgaccggtggacaagcatggttctgggggtgctgtcaccctagggtagcactgtatgtcccaatgaaggtgggggaacagaggtatacactggatctgttgacttccttgaaggaagtcgaatgatgcacagtcatgggggaagtgggtgcctggagcctgacaaaagaagtctcttcagtccaccataaaagacagttgaagaaggatgtggactgggattgtacttttatttatggtgacagtgactaccgggatagtggtggtgtggtcttaagtaggacatggaagacaaaaggacctttggagcctagggatgggggttgggattgtgccatctcagaccggatgctatcctggggccacacaatccaggcctcagtctttctgtgggactgaaagccagaacttttggtagattttggagatgtaaaagtaatgacacttgaagaggcaaagaaaaagaagatgtaaaagtgtggccaggtggtgggccaagcagtgtgtgtgtgtgtgtgcatggattaggtggtaggctagcgaatggatgcatggggagatagataatggatggaggataggtagatgatagatagatagatagatagatagatagatagatagatagatagacagacagacagtggatggatggatggatggatggatggatgggcagatagatggacagatgggtggagtggataagtggtagatggggtaaatacatatcaaggataagaatttttatgaccaaatgctctaatgacttatcaaaacaagactatgaggaagccctggatactcaggcaattttgcagatgaggtcgccaagccttaaagacatgaaatcacttgttgtaggtagtggagctgagaactcagccctggcttattccaggcccctggctcatatgttcatattcagttgaattttctcctggtgtgtgctgtgaactggctctgtaaaatgctgacaagatctgggaatcaccttcagctgtgggctacctgggacgtagcttttgctgttgggggggtggcgggggacactgctctgtatctgcggattagtacagagctgctgtgtcacccatattaggagaaactgtgtgactatggggctccgttttaccactcatgtctacactggctgcagtagggtagctggatggtggctctgaccctccttgggcaaagcaacctcttactcagcaagactcaggctggccaagtatgtgggtaggctgggagaaatacactccagcgcttgcctgaggctctcccgttattgtgacccctttctcgatgtgggagggtccctgagtgagctatttagagtacataaaaactatatgttccgagcacacaaaagaggcattcggtgtgtggtgggtgggttgacaagattcgtttgtggaagtccttgaagctatcgccgcggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctaggctgcctctcaagtaggtcaagtggttaagggcctataggggtccccactcccctcttctccaggtaggagccaggttagatctggggcatctgtgtgcctgcagcttgccccaaaccacatacctcatggtttccatcaatggctgggtgccacttaaccctgacctggccccagacctcctccttcatctggtgacagtatccgggagtccagcaaaagccctgctgcccaggtctgctgtatccatattccaaggggatagaccctgcaggtccctacccctgtaaatcacctgccagcctcgcagccggcaccctcgcacccacccacgcttaatggatatggagacattgagccacgcgtcttcttggctagagagctctgagcagcctctcccgagctggccagactggcagtctacagattatccgcttatctgtacagctgctgcaactaaaattcccacagattggagcaaacaattgagctttagagggaccccagaggaagttgtcctggcttccctcacatcgttggagtcctgtatcctccacataagtgtcctgtatcctaacacacacagacagacagacagacagacagacagacagacagatagatagagacagacagacagacagacagacagacatgtgtgtatgttttcctggggccacaataacagtgtctggtgttttgaaagcagctaaaatcttagctaacttttctttgcctgagaccatctctacctcccaagtattcctcacctataaagaaaattcaaaagacctcaagaagaccccaggattacttccactgcagccaagtggccaggcgcaaggacgggcttggggaggggctggggtgcagattctcattgttcttcctatgctgaggcagagggtcaggtaagaggaggctggcaacaggcctgtatgctaatgcgcaccacacagtagggatctttagcgtgtcacctgaccaaaatcacgaaaggaaggcgcatgctgtaaaccacgaggtcagaaaccaaatctcaagtggatagtgacacagtctccgtgttacagatttcgcacgaaatgtctccctaatcttagttagcaagtaggaactggtctgaacagttagttctaaagcagcatgctcgggcatgtttagatagctggctgtaatttgaccatacttcctgaagtggtcctagctagatgccaaaatcactccaactgtcccatggaatccagctgttgattgtgaccctgtgatgatcataaccagatgaagcag

SEQ ID NO: 20 - DNA, 997 bp, Mus musculus
  misc_feature (384)..(997): SCE1 Sequence
gtatctgcggattagtacagagctgctgtgtcacccatattaggagaaactgtgtgactatggggctccgttttaccactcatgtctacactggctgcagtagggtagctggatggtggctctgaccctccttgggcaaagcaacctcttactcagcaagactcaggctggccaagtatgtgggtaggctgggagaaatacactccagcgcttgcctgaggctctcccgttattgtgacccctttctcgatgtgggagggtccctgagtgagctatttagagtacataaaaactatatgttccgagcacacaaaagaggcattcggtgtgtggtgggtgggttgacaagattcgtttgtggaagtccttgaagctatcgccgcggcagcctggccggcagccacatgcctttcatagatgcagaatttctgtcatagcaagtccacaggctacaccatgggcctttttgtttcctgtgccctcccaggtgaccccaagcccaggctgccagcggcagaagtattccatgaacaaggaaggtcctctctagccgggcccacacgcccagattccatagctcctctgcaggcctaggctgcctctcaagtaggtcaagtggttaagggcctataggggtccccactcccctcttctccaggtaggagccaggttagatctggggcatctgtgtgcctgcagcttgccccaaaccacatacctcatggtttccatcaatggctgggtgccacttaaccctgacctggccccagacctcctccttcatctggtgacagtatccgggagtccagcaaaagccctgctgcccaggtctgctgtatccatattccaaggggatagaccctgcaggtccctacccctgtaaatcacctgccagcctcgcagccggcaccctcgcacccacccacgcttaatggatatggagacattgagccacgcgtcttcttggctagagagctctgagcagcctctcccgagc

SEQ ID NO: 21 - DNA, 1022 bp, Artificial sequence
  Artificial Sequence = alternative consensus sequence from human/mouse Mod4
gtmwctgygrawtwswmcaraggctgyrymacctarsmgaawctryrtgamtatkgggctcyktwwkaaycwctyatstctacrcyrrcygyrktagggwagctgggcatgkkgrctstsaccsysctkaaggrsraagsaacctgagctyastswgyrcagmctcacaggggtgrctggccagyryrtggsyrsgcyksgmsramymcryyccmrcsctcctgaggctcyccyrkyayacggctgcctctcctgtgrccycttyctgmygtgggaggkkccmkgrgtgagctatktrgagyamataaaaactatatgttcygrgcacacaaargaggcattggtgtgwgrtgggtgggttgayragmytcgtttgtgggaarkyyttsaagmwatckcygcggsaagcctggcmggcagccacatgcctytcatagatgcagaatttctgycatmgcaartccacaggcyacaccatggscctttttgtttcctktgccctcccwggtgaccccaagcmcagssygccgygrcasaagtattccayraacaaggarsgtcctctytrgccstggcccacackyccarattccatrgyctctgcaggsctmggcykcctykmragrggycargwgrktrrrggccywkmgkgsycccsrckccttctccagkkrgrmrmsrggyyasakctkrrrsmtckgttsysyrcmkytwgccccaarcymymywcmycwysryyyccakmaawsrcactkgktgycrytkmmmyckswcmwrgyyycwgwccgayyctycwymrkcwggkrrcagactatcyrkswgyysrrcarragcyskrsyksmswgstckrcwrwakccmtrgytccmaskggryasascctgcargycyctmcccmtsyatgagagarrtcacctgcsrsccwckcwgcmgscasystmsmrccyryccamgstkrmyrratrkrgagrcaykgaccasgmgycywytwgrsymrrsagmkctgmrcrgscwmtsccswsc

SEQ ID NO: 22 - DNA, 997 bp, Artificial sequence
  Artificial Sequence = alternative consensus sequence from mouse/human Mod4
gtmwctgygrawtwswmcaragctgctgyrymacccatattarsmgaawctryrtgamtatkgggctcyktwwkaycwctyatstctacrcyrrcygyrktagggwagctggatgkkgrctstsaccsysctkggrsraagsaacctctyastswgyragmctcagrctggccaagyryrtggsyrsgcyksgmsramymcryyccmrcscttgcctgaggctcyccyrkyaytgtgrccycttyctcgmygtgggaggkkccmkgrgtgagctatktrgagyamataaaaactatatgttcygrgcacacaaargaggcattcggtgtgwgrtgggtgggttgayragmytcgtttgtggaarkyyttsaagmwatckcygcggsagcctggcmggcagccacatgcctytcatagatgcagaatttctgycatmgcaartccacaggcyacaccatggscctttttgtttcctktgccctcccwggtgaccccaagcmcagssygccagygrcasaagtattccayraacaaggarsgtcctctytrgccsggcccacackyccarattccatrgytcctctgcaggsctmggcykcctykmragtrggycargwgrktrrrggccywkmgkgsycccsrckcccctcttctccagkkrgrmrmsrggyyasakctkrrrsmtckgtgtsysyrcmkytwgccccaarcymymywcmycwysryyyccakmaawsrctkgktgycrytkmmmyckswcmwrgyyycwgwccyyctycwymrkcwggkrrcagtatcyrkswgyysrrcarragcyskrsyksmswgstckrcwrwakccmtrytccmaskggryasascctgcargycyctmcccmtsyarrtcacctgcsrsccwckcwgcmgscasystmsmrccyryccamgstkrmyrratrkrgagrcaykgagccasgmgycywytwgrsymrrsagmkctgmrcrgscwmtsccswsc

SEQ ID NO: 23 - DNA, 484 bp, Artificial sequence
  Artificial Sequence = alternative consesus sub-sequence from mouse/human SCE1
cggsagcctggcmggcagccacatgcctytcatagatgcagaatttctgycatmgcaartccacaggcyacaccatggscctttttgtttcctktgccctcccwggtgaccccaagcmcagssygccagygrcasaagtattccayraacaaggarsgtcctctytrgccsggcccacackyccarattccatrgytcctctgcaggsctmggcykcctykmragtrggycargwgrktrrrggccywkmgkgsycccsrckcccctcttctccagkkrgrmrmsrggyyasakctkrrrsmtckgtgtsysyrcmkytwgccccaarcymymywcmycwysryyyccakmaawsrctkgktgycrytkmmmyckswcmwrgyyycwgwccyyctycwymrkcwggkrrcagtatcyrkswgyysrrcarragcyskrsyksmswgstckrcwrwakccmtrytccmaskggryasascctgca